SEQUENCE LISTING (87 sequences)

SEQ ID NO: 1 - protein, 332 aa, Homo sapiens
  hLDH-M subunit (also referred to LDH-5)
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 2 - protein, 334 aa, Homo sapiens
  hLDH-H subunit (also referred to LDH-1)
MATLKEKLIAPVAEEEATVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 3 - protein, 312 aa, Artificial Sequence
  hLDH-Mtr
NKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 4 - protein, 314 aa, Artificial Sequence
  hLDH-Htr
NNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 5 - protein, 16 aa, Artificial Sequence
  GP-X
  VARIANT 2: Amino acid residue at position 2 represents E, D, or A
  VARIANT 5: Amino acid residue at position 5 represents D, E, or A
  VARIANT 11: Amino acid residue at position 11 represents L, A, V, I, F, W, or Y
  VARIANT 12: Amino acid residue at position 12 represents F, A, L, V, I, W, or Y
  VARIANT 13: Amino acid residue at position 13 represents L, A, V, I, F, W, or Y
GXMMXLQHGSXXXQTP

SEQ ID NO: 6 - protein, 16 aa, Artificial Sequence
  GP-16
GEMMDLQHGSLFLQTP

SEQ ID NO: 7 - protein, 16 aa, Artificial Sequence
  GP-16/E62D
GDMMDLQHGSLFLQTP

SEQ ID NO: 8 - protein, 16 aa, Artificial Sequence
  GP-16/E62A
GAMMDLQHGSLFLQTP

SEQ ID NO: 9 - protein, 16 aa, Artificial Sequence
  GP-16/D65E
GEMMELQHGSLFLQTP

SEQ ID NO: 10 - protein, 16 aa, Artificial Sequence
  GP-16/D65A
GEMMALQHGSLFLQTP

SEQ ID NO: 11 - protein, 16 aa, Artificial Sequence
  GP-16/L71A
GEMMDLQHGSAFLQTP

SEQ ID NO: 12 - protein, 16 aa, Artificial Sequence
  GP-16/L71V
GEMMDLQHGSVFLQTP

SEQ ID NO: 13 - protein, 16 aa, Artificial Sequence
  GP-16/L71I
GEMMDLQHGSIFLQTP

SEQ ID NO: 14 - protein, 16 aa, Artificial Sequence
  GP-16/L71F
GEMMDLQHGSFFLQTP

SEQ ID NO: 15 - protein, 16 aa, Artificial Sequence
  GP-16/L71W
GEMMDLQHGSWFLQTP

SEQ ID NO: 16 - protein, 16 aa, Artificial Sequence
  GP-16/L71Y
GEMMDLQHGSYFLQTP

SEQ ID NO: 17 - protein, 16 aa, Artificial Sequence
  GP-16/F72A
GEMMDLQHGSLALQTP

SEQ ID NO: 18 - protein, 16 aa, Artificial Sequence
  GP-16/F72L
GEMMDLQHGSLLLQTP

SEQ ID NO: 19 - protein, 16 aa, Artificial Sequence
  GP-16/F72V
GEMMDLQHGSLVLQTP

SEQ ID NO: 20 - protein, 16 aa, Artificial Sequence
  GP-16/F72I
GEMMDLQHGSLILQTP

SEQ ID NO: 21 - protein, 16 aa, Artificial Sequence
  GP-16/F72W
GEMMDLQHGSLWLQTP

SEQ ID NO: 22 - protein, 16 aa, Artificial Sequence
  GP-16/F72Y
GEMMDLQHGSLYLQTP

SEQ ID NO: 23 - protein, 16 aa, Artificial Sequence
  GP-16/L73A
GEMMDLQHGSLFAQTP

SEQ ID NO: 24 - protein, 16 aa, Artificial Sequence
  GP-16/L73V
GEMMDLQHGSLFVQTP

SEQ ID NO: 25 - protein, 16 aa, Artificial Sequence
  GP-16/L73I
GEMMDLQHGSLFIQTP

SEQ ID NO: 26 - protein, 16 aa, Artificial Sequence
  GP-16/L73F
GEMMDLQHGSLFFQTP

SEQ ID NO: 27 - protein, 16 aa, Artificial Sequence
  GP-16/L73W
GEMMDLQHGSLFWQTP

SEQ ID NO: 28 - protein, 16 aa, Artificial Sequence
  GP-16/L73Y
GEMMDLQHGSLFYQTP

SEQ ID NO: 29 - protein, 22 aa, Artificial Sequence
  LP-22
LEDKLKGEMMDLQHGSLFLQTP

SEQ ID NO: 30 - protein, 22 aa, Artificial Sequence
  LP-22/E62D
LEDKLKGDMMDLQHGSLFLQTP

SEQ ID NO: 31 - protein, 22 aa, Artificial Sequence
  LP-22/E62A
LEDKLKGAMMDLQHGSLFLQTP

SEQ ID NO: 32 - protein, 22 aa, Artificial Sequence
  LP-22/D65E
LEDKLKGEMMELQHGSLFLQTP

SEQ ID NO: 33 - protein, 22 aa, Artificial Sequence
  LP-22/D65A
LEDKLKGEMMALQHGSLFLQTP

SEQ ID NO: 34 - protein, 22 aa, Artificial Sequence
  LP-22/L71A
LEDKLKGEMMDLQHGSAFLQTP

SEQ ID NO: 35 - protein, 22 aa, Artificial Sequence
  LP-22/L71V
LEDKLKGEMMDLQHGSVFLQTP

SEQ ID NO: 36 - protein, 22 aa, Artificial Sequence
  LP-22/L71I
LEDKLKGEMMDLQHGSIFLQTP

SEQ ID NO: 37 - protein, 22 aa, Artificial Sequence
  LP-22/L71F
LEDKLKGEMMDLQHGSFFLQTP

SEQ ID NO: 38 - protein, 22 aa, Artificial Sequence
  LP-22/L71W
LEDKLKGEMMDLQHGSWFLQTP

SEQ ID NO: 39 - protein, 22 aa, Artificial Sequence
  LP-22/L71Y
LEDKLKGEMMDLQHGSYFLQTP

SEQ ID NO: 40 - protein, 22 aa, Artificial Sequence
  LP-22/F72A
LEDKLKGEMMDLQHGSLALQTP

SEQ ID NO: 41 - protein, 22 aa, Artificial Sequence
  LP-22/F72L
LEDKLKGEMMDLQHGSLLLQTP

SEQ ID NO: 42 - protein, 22 aa, Artificial Sequence
  LP-22/F72V
LEDKLKGEMMDLQHGSLVLQTP

SEQ ID NO: 43 - protein, 22 aa, Artificial Sequence
  LP-22/F72I
LEDKLKGEMMDLQHGSLILQTP

SEQ ID NO: 44 - protein, 22 aa, Artificial Sequence
  LP-22/F72W
LEDKLKGEMMDLQHGSLWLQTP

SEQ ID NO: 45 - protein, 22 aa, Artificial Sequence
  LP-22/F72Y
LEDKLKGEMMDLQHGSLYLQTP

SEQ ID NO: 46 - protein, 22 aa, Artificial Sequence
  LP-22/L73A
LEDKLKGEMMDLQHGSLFAQTP

SEQ ID NO: 47 - protein, 22 aa, Artificial Sequence
  LP-22/L73V
LEDKLKGEMMDLQHGSLFVQTP

SEQ ID NO: 48 - protein, 22 aa, Artificial Sequence
  LP-22/L73I
LEDKLKGEMMDLQHGSLFIQTP

SEQ ID NO: 49 - protein, 22 aa, Artificial Sequence
  LP-22/L73F
LEDKLKGEMMDLQHGSLFFQTP

SEQ ID NO: 50 - protein, 22 aa, Artificial Sequence
  LP-22/L73W
LEDKLKGEMMDLQHGSLFWQTP

SEQ ID NO: 51 - protein, 22 aa, Artificial Sequence
  LP-22/L73Y
LEDKLKGEMMDLQHGSLFYQTP

SEQ ID NO: 52 - protein, 334 aa, Artificial Sequence
  hLDH-1/G61A
MATLKEKLIAPVAEEEATVPNNKITVVGVGQVGMACAISILGKSLADELALVDVLEDKLKAEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 53 - protein, 334 aa, Artificial Sequence
  hLDH-1/E62A
MATLKEKLIAPVAEEEATVPNNKITVVGVGQVGMACAISILGKSLADELALVDVLEDKLKGAMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 54 - protein, 334 aa, Artificial Sequence
  hLDH-1/M63A
MATLKEKLIAPVAEEEATVPNNKITVVGVGQVGMACAISILGKSLADELALVDVLEDKLKGEAMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 55 - protein, 334 aa, Artificial Sequence
  hLDH-1/M64A
MATLKEKLIAPVAEEEATVPNNKITVVGVGQVGMACAISILGKSLADELALVDVLEDKLKGEMADLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 56 - protein, 334 aa, Artificial Sequence
  hLDH-1/D65A
MATLKEKLIAPVAEEEATVPNNKITVVGVGQVGMACAISILGKSLADELALVDVLEDKLKGEMMALQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 57 - protein, 334 aa, Artificial Sequence
  hLDH-1/L66A
MATLKEKLIAPVAEEEATVPNNKITVVGVGQVGMACAISILGKSLADELALVDVLEDKLKGEMMDAQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 58 - protein, 334 aa, Artificial Sequence
  hLDH-1/Q67A
MATLKEKLIAPVAEEEATVPNNKITVVGVGQVGMACAISILGKSLADELALVDVLEDKLKGEMMDLAHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 59 - protein, 334 aa, Artificial Sequence
  hLDH-1/H68A
MATLKEKLIAPVAEEEATVPNNKITVVGVGQVGMACAISILGKSLADELALVDVLEDKLKGEMMDLQAGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 60 - protein, 334 aa, Artificial Sequence
  hLDH-1/G69A
MATLKEKLIAPVAEEEATVPNNKITVVGVGQVGMACAISILGKSLADELALVDVLEDKLKGEMMDLQHASLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 61 - protein, 334 aa, Artificial Sequence
  hLDH-1/S70A
MATLKEKLIAPVAEEEATVPNNKITVVGVGQVGMACAISILGKSLADELALVDVLEDKLKGEMMDLQHGALFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 62 - protein, 334 aa, Artificial Sequence
  hLDH-1/L71A
MATLKEKLIAPVAEEEATVPNNKITVVGVGQVGMACAISILGKSLADELALVDVLEDKLKGEMMDLQHGSAFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 63 - protein, 334 aa, Artificial Sequence
  hLDH-1/F72A
MATLKEKLIAPVAEEEATVPNNKITVVGVGQVGMACAISILGKSLADELALVDVLEDKLKGEMMDLQHGSLALQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 64 - protein, 334 aa, Artificial Sequence
  hLDH-1/L73A
MATLKEKLIAPVAEEEATVPNNKITVVGVGQVGMACAISILGKSLADELALVDVLEDKLKGEMMDLQHGSLFAQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 65 - protein, 334 aa, Artificial Sequence
  hLDH-1/Q74A
MATLKEKLIAPVAEEEATVPNNKITVVGVGQVGMACAISILGKSLADELALVDVLEDKLKGEMMDLQHGSLFLATPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 66 - protein, 334 aa, Artificial Sequence
  hLDH-1/T75A
MATLKEKLIAPVAEEEATVPNNKITVVGVGQVGMACAISILGKSLADELALVDVLEDKLKGEMMDLQHGSLFLQAPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 67 - protein, 334 aa, Artificial Sequence
  hLDH-1/P76A
MATLKEKLIAPVAEEEATVPNNKITVVGVGQVGMACAISILGKSLADELALVDVLEDKLKGEMMDLQHGSLFLQTAKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 68 - protein, 15 aa, Artificial Sequence
  Antennapedia Penetratin CCP
RQIKWFQNRRMKWKK

SEQ ID NO: 69 - protein, 11 aa, Artificial Sequence
  TAT CCP
YGRKKRRQRRR

SEQ ID NO: 70 - protein, 18 aa, Artificial Sequence
  SynB1 CCP
RGGRLSYSRRRFSTSTGR

SEQ ID NO: 71 - protein, 10 aa, Artificial Sequence
  SynB3 CCP
RRLSYSRRRF

SEQ ID NO: 72 - protein, 12 aa, Artificial Sequence
  PTD-4 CCP
PIRRRKKLRRLK

SEQ ID NO: 73 - protein, 12 aa, Artificial Sequence
  PTD-5 CCP
RRQRRTSKLMKR

SEQ ID NO: 74 - protein, 15 aa, Artificial Sequence
  FHV Coat-(35-49) CCP
RRRRNRTRRNRRRVR

SEQ ID NO: 75 - protein, 19 aa, Artificial Sequence
  BMV Gag-(7-25) CCP
KMTRAQRRAAARRNRWTAR

SEQ ID NO: 76 - protein, 13 aa, Artificial Sequence
  HTLV-II Rex-(4-16) CCP
TRRQRTRRARRNR

SEQ ID NO: 77 - protein, 13 aa, Artificial Sequence
  D-Tat CCP
GRKKRRQRRRPPQ

SEQ ID NO: 78 - protein, 13 aa, Artificial Sequence
  R9-Tat CCP
GRRRRRRRRRPPQ

SEQ ID NO: 79 - protein, 27 aa, Artificial Sequence
  Transportan CCP
GWTLNSAGYLLGKINLKALAALAKKIL

SEQ ID NO: 80 - protein, 17 aa, Artificial Sequence
  MAP CCP
KLALKLALKLALALKLA

SEQ ID NO: 81 - protein, 27 aa, Artificial Sequence
  SBP CCP
MGLGLHLLVLAAALQGAWSQPKKKRKV

SEQ ID NO: 82 - protein, 27 aa, Artificial Sequence
  FBP CCP
GALFLGWLGAAGSTMGAWSQPKKKRKV

SEQ ID NO: 83 - protein, 27 aa, Artificial Sequence
  MPG CCP
GALFLGFLGAAGSTMGAWSQPKKKRKV

SEQ ID NO: 84 - protein, 27 aa, Artificial Sequence
  MPG(DeltaNLS) CCP
GALFLGFLGAAGSTMGAWSQPKSKRKV

SEQ ID NO: 85 - protein, 21 aa, Artificial Sequence
  PEP-1 CCP
KETWWETWWTEWSQPKKKRKV

SEQ ID NO: 86 - protein, 21 aa, Artificial Sequence
  PEP-2 CCP
KETWFETWFTEWSQPKKKRKV

SEQ ID NO: 87 - protein, 17 aa, Artificial Sequence
  PEP-17
GEMMDLQHGSLFLQTPK

(19) United States
(12) Patent Application Publication
(10) Pub. No.: US 2024/0116987 A1
(43) Pub. Date: Apr. 11, 2024

(54) POLYPEPTIDE INHIBITORS OF LACTATE DEHYDROGENASE ACTIVITY FOR USE IN CANCER THERAPY

(71) Applicant: UNIVERSITÉ CATHOLIQUE DE LOUVAIN, Louvain-la-Neuve (BE)

(72) Inventors: Pierre SONVEAUX, Sterrebeek (BE); Raphaël FREDERICK, Godinne (BE); Léopold THABAULT, London (GB); Maxime LIBERELLE, Loos (FR)

(73) Assignee: UNIVERSITÉ CATHOLIQUE DE LOUVAIN, Louvain-la-Neuve (BE)

(21) Appl. No.: 18/263,756

(22) PCT Filed: Feb. 1, 2022
(86) PCT No.: PCT/EP2022/052282

§ 371 Date: Aug. 1, 2023

(30) Foreign Application Priority Data
Feb. 1, 2021 (EP) 21154636.1

(51) Int. Cl. C07K 7/08

(52) U.S. Cl. CPC C07K 7/08

(57) ABSTRACT

A polypeptide including the amino acid sequence of formula (I): GX1MMX2LQHGSX3X4X5QTP. These polypeptides modulate the activity of the native tetrameric lactate dehydrogenase LDH-1, by inhibiting the tetramerization of its subunits. Also, the therapeutic use of these polypeptides as a medicament, in particular for the prevention and/or the treatment of cancer.

### FIELD OF INVENTION

The present invention relates to polypeptides that modulate the activity of native tetrameric lactate dehydrogenase, as active agents for cancer therapy. More particularly, the invention relates to polypeptides that inhibit the tetramerization of the lactate dehydrogenase subunits.

### BACKGROUND OF INVENTION

Dysregulation of glucose metabolism is a common feature of most cancer cells. The elevated glycolytic flux in cancer cells has two origins, namely, the adaptation to hypoxia (anaerobic glycolysis) and the adaptation to high proliferation rates (aerobic glycolysis, also known as the “Warburg effect”). This higher glycolytic flux provides cancer cells with the energy and biomass essential for the sustainment of their anabolic growth. At the end of the glycolytic pathway stands the reduction of pyruvate to lactate, which is catalyzed by lactate dehydrogenases (LDHs).

While lactate has long been considered as a mere by-product of glycolysis, it is now regarded as a potential purpose of accelerated glycolysis in cancer, in the light of the numerous benefits it provides to tumor growth. It is now acknowledged that elevation of lactate production indeed promotes several phenomena such as angiogenesis, invasiveness, commensalism, inflammation, as well as redox homeostasis. Lactate metabolism further establishes a metabolic symbiosis between oxidative cancer cells that use lactate preferentially to glucose as a fuel, and glycolytic cancer cells that rapidly convert glucose to lactate. Lactate oxidation to pyruvate by LDHs further promotes lysosomal acidification and autophagy.

LDHs are key enzymes at the core of this adaptive metabolism as they catalyze the terminal reaction of lactate biosynthesis with the interconversion of pyruvate and NADH to lactate and NAD+. LDHs function as obligate tetramers constituted by the homo or hetero association of two isoenzymes, LDH-H (encoded by the LDHB gene) and LDH-M (encoded by the LDHA gene). These two isoenzymes show very high homology and identity. The two LDH homotetramers, LDH-1 (LDH-H4) and LDH-5 (LDH-M4), are the most extensively studied forms of LDHs and constitute appealing targets for cancer therapy.

Intense efforts were initially devoted to selective LDH-5 inhibition due to its broad implication in cancer pathogenesis. However, more recent reports about the implications of LDH-1 in cancer pathogenesis shed light on LDH-1 inhibition. First, LDH-1 was reported to interact with lysosomal vesicular ATPase, thus regulating autophagy, and is essential for metabolic reprogramming through p53 and Ras mutations (Brisson et aL; Lactate Dehydrogenase B Controls Lysosome Activity and Autophagy in Cancer. Cancer Cell 2016, 30, 418-431; Smith et aL; Addiction to Coupling of the Warburg Effect with Glutamine Catabolism in Cancer Cells. Cell Rep. 2016, 17 (3), 821-836). Second, the LDHB gene was identified to be essential for triple-negative breast cancer (McCleland et aL; An Integrated Genomic Screen Identifies LDHB as an Essential Gene for Triple-Negative Breast Cancer. Cancer Res. 2012, 72 (22), 5812-5823). Finally, it has been shown that one LDH isoenzyme can compensate for the genetic disruption of the other in order to sustain the Warburg phenotype (Ždralevid et al.; Disrupting the ‘Warburg Effect’ Re-Routes Cancer Cells to OXPHOS Offering a Vulnerability Point via ‘Ferroptosis’-Induced Cell Death. Adv. Biol. Regul. 2018, 68, 55-63). Altogether, these studies support the idea that dual LDH inhibitors could bring an additional therapeutic value over selective isoenzyme inhibition.

The therapeutic interest for LDH inhibition prompted the development of potent, dual or selective, active-site LDH inhibitors. However, despite intense efforts, pharmacological LDH inhibition struggled to translate to in vivo activity. In fact, LDHs are usually recognized as poorly druggable targets, and different reasons can account for this. First, LDH active-site inhibitors face a challenge in achieving selectivity over other dehydrogenases, notably due to a common NAD-binding domain. For instance, gossypol derivatives, that are among the first reported LDH inhibitors, demonstrated significant inhibition towards other dehydrogenases. Second, the LDH catalytic-site presents non-optimal physicochemical properties with high solvent exposure and hydrophilicity, leading to challenging absorption, distribution, metabolization and excretion (ADME) properties for most LDH active site inhibitors. Finally, an inherent difficulty in achieving therapeutic LDH inhibition stems from its high intracellular concentration; LDHs are indeed highly concentrated in cancer cells, with protein concentrations reported in the μM range. This high cellular concentration often hampers the observation of cell-based inhibition below that μM threshold, even for the more potent nanomolar inhibitors reaching micromolar concentrations in tumors.

These different challenges to LDH inhibition called for developing new strategies to target this enzymatic family of high therapeutic potential. To this end, tool compounds able to target the LDH oligomeric interface instead of its active site have been recently developed. Targeting a protein oligomeric state is still an underexplored strategy that can provide several benefits over active-site targeting, and could thus overcome the existing difficulties encountered with LDH orthosteric inhibitors. Targeting LDH self-assembly could indeed lead to the identification of new and potentially more druggable allosteric sites. Noteworthy, as LDH subunits can form homo- and heterotetramers, the tetrameric interface is shared between the two different isoenzymes. Targeting LDH tetrameric interface can thus yield to molecules disrupting both LDH-1 and LDH-5, which is in line with the current pan-LDH inhibition strategy. Moreover, disruptors of protein self-assembly can induce protein misfolding and degradation. Therefore, targeting the LDH oligomeric state could reduce its intracellular concentration, leading to sub-stoichiometric inhibition, hence higher efficacy.

To this end, it was previously developed and characterized a dimeric model of LDH-H by truncating its N-terminal tetramerization domain (LDH-Htr) (Thabault et aL; Interrogating the Lactate Dehydrogenase Tetramerization Site Using (Stapled) Peptides. J. Med. Chem. 2020, 63 (9), 4628-464). This model allows to study the LDH tetrameric interface, and previously led to the identification of a first allosteric site and to the generation of linear and cyclic polypeptides based on LDHA and LDHB subunits acting as LDH tetramerization inhibitors for treating cancer(see, e.g., WO2020221899). Another example is the in-silico design and generation of peptides that mimics the N-terminal domain of LDHA, preventing the interaction between the N-terminal and C-terminal regions of the enzyme, thus inhibiting its tetramerization (Jafary et al., Novel Peptide Inhibitors for Lactate Dehydrogenase A (LDHA): a Survey to Inhibit LDHA Activity via Disruption of Protein-Protein interaction, Scientific Reports, 2019).

Novel LDH inhibitors are the subject matter of the present invention.

### SUMMARY

A first aspect of the invention relates to a polypeptide that inhibits the tetramerization of the LDH subunits, the polypeptide comprising the amino acid sequence of formula (I):

(I)
(SEQ ID NO: 5)
GX₁MMX₂LQHGSX₃X₄X₅QTP

wherein:
X₁ represents amino acid residue E, D, or A;
X₂ represents amino acid residue D, E, or A;
X₃ represents amino acid residue L, A, V, I, F, W, or Y;
X₄ represents amino acid residue F, A, L, V, I, W, or Y;
X₅ represents amino acid residue L, A, V, I, F, W, or Y,
wherein said polypeptide comprises from 16 to 200 amino acid residues, and wherein said amino acid sequence is not SEQ ID NO: 87.

In some embodiments, the polypeptide comprises an amino acid sequence selected from the group consisting of SEQ ID NO: 6 to SEQ ID NO: 51.

In certain embodiments, the polypeptide comprises an amino acid sequence selected from the group consisting of SEQ ID NO: 6 to SEQ ID NO: 28.

A further aspect of the invention pertains to a nucleic acid encoding a polypeptide according to the instant invention.

Another aspect of the invention relates to a nucleic acid vector comprising at least one nucleic acid according to the instant invention.

In one aspect, the invention relates to a pharmaceutical composition comprising (i) at least one polypeptide, at least one nucleic acid, or at least one nucleic acid vector according to the instant invention, and (ii) at least one pharmaceutically acceptable vehicle.

A further aspect of the invention relates to a kit comprising (i) at least one polypeptide, at least one nucleic acid, at least one nucleic acid vector, or at least one pharmaceutical composition according to the instant invention, and (ii) at least a means to administer the polypeptide, the nucleic acid, the nucleic acid vector, or the pharmaceutical composition.

In some embodiments, the kit further comprises an anticancer agent.

A still further aspect of the invention relates to a polypeptide, a nucleic acid, a nucleic acid vector, or a pharmaceutical composition according to the instant invention, for use as a medicament.

In certain embodiments, the polypeptide, nucleic acid, nucleic acid vector, or pharmaceutical composition according to the invention are for use in preventing and/or treating cancer.

The invention also pertains to the use of a polypeptide, nucleic acid, nucleic acid vector, or pharmaceutical composition according to the instant invention, for inhibiting the tetramerization of the lactate dehydrogenase subunits.

In some embodiments, the lactate dehydrogenase subunits are LDH-1 subunits and/or LDH-5 subunits.

In certain embodiments, the lactate dehydrogenase subunits are LDH-1 subunits.

In another aspect, the invention relates to a method for preventing and/or treating cancer in an individual in need thereof, comprising at least the step of administering to the individual a therapeutically efficient amount of a polypeptide, nucleic acid, nucleic acid vector, or pharmaceutical composition according to the instant invention.

### Definitions

In the present invention, the following terms have the following meanings:
“About”, when preceding a figure, means plus or less 10% of the value of said figure.
“Amino acid substitution” refers to the replacement in a polypeptide of one amino acid with another amino acid. In one embodiment, an amino acid is replaced with another amino acid having similar structural and/or chemical properties, e.g., conservative amino acid replacements. “Conservative amino acid substitution” may be made on the basis of similarity in polarity, charge, solubility, hydrophobicity, hydrophilicity, and/or the amphipathic nature of the residues involved. For example, nonpolar (hydrophobic) amino acids include alanine (A), leucine (L), isoleucine (I), valine (V), proline (P), phenylalanine (F), tryptophan (W), and methionine (M); polar neutral amino acids include glycine (G), serine (S), threonine (T), cysteine (C), tyrosine (Y), asparagine (N), and glutamine (Q); positively charged (basic) amino acids include arginine (R), lysine (K), and histidine (H); negatively charged (acidic) amino acids include aspartic acid (D) and glutamic acid (E). Non-conservative substitutions will entail exchanging a member of one of these classes for another class. For example, amino acid substitutions can also result in replacing one amino acid with another amino acid having different structural and/or chemical properties, for example, replacing an amino acid from one group (e.g., polar) with another amino acid from a different group (e.g., basic). Amino acid substitutions can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.
“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.
“Nucleic acid” or “polynucleotide” refers to any polyribonucleotide or polydeoxyribonucleotide, which may be unmodified RNA or DNA or modified RNA or DNA. “Nucleic acid” or “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “Nucleic acid” or “polynucleotide” refers to triple-stranded regions comprising RNA or DNA or both RNA and DNA. The term “nucleic acid” or “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “nucleic acid” or “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.
“Preventing cancer” is intended to mean keeping from happening at least one adverse effect or symptom of a cancer.
“Treating cancer” or “treatment” or “alleviation” refers to both therapeutic treatment and prophylactic or preventative measures; wherein the object is to prevent or slow down (lessen) cancer. Those in need of treatment include those already with cancer as well as those prone to have cancer or those in whom cancer is to be prevented. An individual or mammal is successfully “treated” for cancer if, after receiving a therapeutic amount of a polypeptide according to the present invention, the individual shows observable and/or measurable reduction in or absence of one or more of the following: reduction in the number of cancer cells; reduction in the percent of total cells that are cancerous; and/or relief to some extent, one or more of the symptoms associated with cancer, reduced morbidity and mortality, and improvement in quality of life issues. The above parameters for assessing successful treatment and improvement in the cancer are readily measurable by routine procedures familiar to a physician.
“Therapeutically effective amount” is intended to refer to the level or amount of agent that is aimed at, without causing significant negative or adverse side effects to the target, (1) delaying or preventing the onset of cancer, (2) slowing down or stopping the progression, aggravation, or deterioration of one or more symptoms of cancer; (3) bringing about ameliorations of the symptoms of cancer; (4) reducing the severity or incidence of cancer, or (5) preventing cancer formation. In one embodiment, a therapeutically effective amount is administered prior to the onset of cancer formation, for a prophylactic or preventive action.
“Individual” refers to an animal, preferably a mammal, more preferably a human. In one embodiment, the individual is a man. In another embodiment, the individual is a woman. In one embodiment, an individual may be a “patient”, i.e. a warm-blooded animal, more preferably a human, who/which is awaiting the receipt of, or is receiving medical care or was/is/will be the object of a medical procedure, or is monitored for the development of cancer. In one embodiment, the individual is an adult (for example a subject above the age of 18). In another embodiment, the individual is a child (for example a subject below the age of 18).

### DETAILED DESCRIPTION

This invention relates to polypeptides that modulate the activity of at least one isoform of the native tetrameric lactate dehydrogenase. The inventors herein report the existence of a newly identified allosteric site, allowing the development of a new family of polypeptides functioning as LDH inhibitors.

By “lactate dehydrogenase” or “LDH”, it is meant a tetrameric enzyme that is capable of catalyzing the interconversion of pyruvate and lactate with concomitant interconversion of NADH and NAD.

To date, 5 isoforms of lactate dehydrogenase, i.e., LDH-1, LDH-2, LDH-3, LDH-4 and LDH-5, have been identified, which account for a peculiar combination of 2 subunits, namely the LDH-A subunit and the LDH-B subunit.

Within the context of the invention, by “modulating”, it is meant that the polypeptide of the invention has a biological effect of significantly up-regulating or down-regulating the biological activity of any one of the 5 isoforms of the lactate dehydrogenase, i.e., LDH-1, LDH-2, LDH-3, LDH-4 and LDH-5 and or the biological activity of one or more subunit(s), i.e. the LDH-H subunit and/or the LDH-M subunit.

By “native”, it is meant that the sequence of lactate dehydrogenase (LDH), as referred to in the present application, is derived from nature, e.g., from any species. Further, such native sequence of lactate dehydrogenase can be isolated from nature or can be produced by recombinant or synthetic means from subunit LDH-H and/or LDH-M.

In some embodiments, the LDH-M subunit is represented by an amino acid sequence SEQ ID NO: 1, and the LDH-H subunit is represented by an amino acid sequence SEQ ID NO: 2.

In a particular embodiment, the polypeptide of the invention inhibits the activity of at least one isoform of the native tetrameric lactate dehydrogenase or at least one subunit thereof. In a particular aspect, the invention relates to polypeptide inhibitors of the activity of at least one isoform of the native tetrameric lactate dehydrogenase or at least one subunit thereof.

By “Inhibitor” or “inhibiting”, it is meant that the polypeptide of the invention has for biological effect to inhibit or significantly reduce or down-regulate the biological activity of any one of the 5 isoforms of lactate dehydrogenase. In a particular embodiment, the polypeptide according to the invention is capable of inhibiting up to about 10%, preferably up to about 25%, preferably up to about 50%, preferably up to about 75%, 80%, 90%, 95%, more preferably up to about 96%, 97%, 98%, 99% or 100% of the activity of the native lactate dehydrogenase.

In an embodiment, the polypeptide of the invention inhibits the tetramerization of the lactate dehydrogenase subunits.

In some embodiment, the polypeptide of the invention inhibits the tetramerization of at least one of the 4 LDH-M subunits, so as to inhibit the activity of isoform LDH-5.

In some embodiment, the polypeptide of the invention inhibits the tetramerization of at least one of the 3 LDH-M subunits and/or the LDH-H subunit, so as to inhibit the activity of isoform LDH-4.

In some embodiment, the polypeptide of the invention inhibits the tetramerization of at least one of the 2 LDH-M subunits and/or at least one of the 2 LDH-H subunits, so as to inhibit the activity of isoform LDH-3.

In some embodiment, the polypeptide of the invention inhibits the tetramerization of the LDH-M subunit and/or at least one of the 3 LDH-H subunits so as to inhibit the activity of isoform LDH-2.

In some embodiment, the polypeptide of the invention inhibits the tetramerization of at least one of the 4 LDH-H subunits, so as to inhibit the activity of isoform LDH-1.

It is needless to mention that the inhibition of the tetramerization of the lactate dehydrogenase subunits may be assessed by any suitable mean available in the state of the art, in particular any suitable biochemical or biophysical method.

Illustratively, biochemical methods, such as, e.g., affinity electrophoresis, bimolecular fluorescence complementation (BiFC), co-immunoprecipitation, tandem affinity purification, intrinsic tryptophan fluorescence, size exclusion chromatography, fractionated centrifugation, cross-linking (SDS PAGE) electrophoresis; or biophysical methods, such as, e.g., biacore, dual polarization interferometry (DPI), dynamic light scattering (DLS), microscale thermophoresis (MST), NMR WaterLOGSY, Saturation Transfer Difference (STD) spectroscopy, Carr Purcell Meiboom Gill (CPMG) pulse sequence and/or static light scattering (SLS), surface plasmon resonance (SPR) may be employed.

In some embodiment, the inhibition of the tetramerization of at least one of the lactate dehydrogenase subunits may be assessed by the ability of the polypeptide according to the invention to bind to one or more LDH subunit(s) lacking the N-terminus 20 amino acid residues, namely, truncated LDH-M or LDH-Mtr, and truncated LDH-H or LDH-Htr.

In some embodiments, the polypeptide of the invention does not bind the N-terminus 20 amino acid residues of LDH-H and/or LDH-M. In some embodiments, the polypeptide of the invention does not bind the N-terminus 15 amino acid residues of LDH-H and/or LDH-M.

In some embodiments, the polypeptide binds at least one amino acid at position 62, 65, 71, 72 or 73 of LDH-H, wherein said amino acid position is defined with respect to SEQ ID NO: 2.

In certain embodiment, LDH-Mtr is represented by an amino acid sequence SEQ ID NO: 3. In some embodiment, LDH-Htr is represented by an amino acid sequence SEQ ID NO: 4.

In some embodiment, when the MST method is implemented, a significant binding of a polypeptide according to the invention to LDH-Mtr (SEQ ID NO: 3) or LDH-Htr (SEQ ID NO: 4), preferably to LDH-Htr (SEQ ID NO: 4), may result in a dissociation constant (Kd) comprised from 1 μM to 5 mM, preferably from about 50 μM to about 3.5 mM.

Within the scope of the invention, the expression “from about 1 μM to about 5 mM” includes 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 1.5 mM, 2 mM, 2.5 mM, 3 mM, 3.5 mM, 4 mM, 4.5 mM and 5 mM.

One aspect of the invention relates to a polypeptide comprising or consisting of the amino acid sequence of formula (I):

(I)
(SEQ ID NO: 5)
GX₁MMX₂LQHGSX₃X₄X₅QTP

wherein:
X₁ represents amino acid residue E, D, or A;
X₂ represents amino acid residue D, E, or A;
X₃ represents amino acid residue L, A, V, I, F, W, or Y;
X₄ represents amino acid residue F, A, L, V, I, W, or Y;
X₅ represents amino acid residue L, A, V, I, F, W, or Y.

In certain embodiments, the polypeptide according to the invention modulates the activity of at least one isoform of the native tetrameric lactate dehydrogenase. In some embodiments, the polypeptide according to the invention inhibits the tetramerization of the lactate dehydrogenase subunits.

A further aspect of the invention relates to a polypeptide that inhibits the tetramerization of the LDH subunits, the 1 tide comprising the amino acid sequence of formula (I):

(I)
(SEQ ID NO: 5)
GX₁MMX₂LQHGSX₃X₄X₅QTP

wherein:
X₁ represents amino acid residue E, D, or A;
X₂ represents amino acid residue D, E, or A;
X₃ represents amino acid residue L, A, V, I, F, W, or Y;
X₄ represents amino acid residue F, A, L, V, I, W, or Y;
X₅ represents amino acid residue L, A, V, I, F, W, or Y.

In certain embodiments, the polypeptide consists of the amino acid sequence of formula (I):

(I)
(SEQ ID NO: 5)
GX₁MMX₂LQHGSX₃X₄X₅QTP

wherein:
X₁ represents amino acid residue E, D, or A;
X₂ represents amino acid residue D, E, or A;
X₃ represents amino acid residue L, A, V, I, F, W, or Y;
X₄ represents amino acid residue F, A, L, V, I, W, or Y;
X₅ represents amino acid residue L, A, V. I, F, W, or Y.

In certain embodiments, the polypeptide comprises of the amino acid sequence of formula (I):

(I)
(SEQ ID NO: 5)
GX₁MMX₂LQHGSX₃X₄X₅QTP

wherein:
X₁ represents amino acid residue E or A;
X₂ represents amino acid residue D or A;
X₃ represents amino acid residue L or A;
X₄ represents amino acid residue F or A;
X₅ represents amino acid residue L or A.

In certain embodiments, the polypeptide consists of the amino acid sequence of formula (I):

(I)
(SEQ ID NO: 5)
GX₁MMX₂LQHGSX₃X₄X₅QTP

wherein:
X₁ represents amino acid residue E or A;
X₂ represents amino acid residue D or A;
X₃ represents amino acid residue L or A;
X₄ represents amino acid residue F or A;
X₅ represents amino acid residue L or A.

In some embodiments, the polypeptide comprises an amino acid sequence selected from the group consisting of SEQ ID NO: 6 to SEQ ID NO: 51, SEQ ID NO: 53, SEQ ID NO: 56, and SEQ ID NO: 62 to SEQ ID NO: 64.

In certain embodiments, the polypeptide consists of an amino acid sequence selected from the group consisting of SEQ ID NO: 6 to SEQ ID NO: 51, SEQ ID NO: 53, SEQ ID NO: 56, and SEQ ID NO: 62 to SEQ ID NO: 64.

In some embodiments, the polypeptide comprises or consists of an amino acid sequence selected from the group consisting of SEQ ID NO: 6 to SEQ ID NO: 51.

In some embodiments, the polypeptide comprises or consists of an amino acid sequence GEMMDLQHGSLFLQTP (SEQ ID NO: 6). In practice, the polypeptide of amino acid sequence GEMMDLQHGSLFLQTP (SEQ ID NO: 6) is referred to as polypeptide GP-16.

In some embodiments, the polypeptide comprises an amino acid sequence selected from the group consisting of SEQ ID NO: 6 to SEQ ID NO: 28.

In some embodiments, the polypeptide consists of an amino acid sequence selected from the group consisting of SEQ ID NO: 6 to SEQ ID NO: 28.

In some embodiments, the polypeptide comprises an amino acid sequence selected from the group consisting of SEQ ID NO: 6, SEQ ID NO: 8, SEQ ID NO: 10, SEQ ID NO: 11, SEQ ID NO: 17 and SEQ ID NO: 23.

In some embodiments, the polypeptide consists of an amino acid sequence selected from the group consisting of SEQ ID NO: 6, SEQ ID NO: 8, SEQ ID NO: 10, SEQ ID NO: 11, SEQ ID NO: 17 and SEQ ID NO: 23.

In certain embodiments, the polypeptide comprises or consists of an amino acid sequence LEDKLKGEMMDLQHGSLFLQTP (SEQ ID NO: 29). In practice, the polypeptide of amino acid sequence LEDKLKGEMMDLQHGSLFLQTP (SEQ ID NO: 29) is referred to as polypeptide LP-22.

In some embodiments, the polypeptide comprises or consists of an amino acid sequence selected from the group consisting of SEQ ID NO: 29 to SEQ ID NO: 51.

In certain embodiments, the polypeptide comprises or consists of an amino acid sequence selected from the group consisting of SEQ ID NO: 31, SEQ ID NO: 33, and SEQ ID NO: 34, SEQ ID NO: 40 and SEQ ID NO: 46.

In some embodiments, the polypeptide comprises or consists of the amino acid sequence of LDH-H (also referred to as LDH-1), i.e., SEQ ID NO: 2, in which one amino acid residue is substituted accordingly: E62A (SEQ ID NO: 53), D65A (SEQ ID NO: 56), L71A (SEQ ID NO: 62), F72A (SEQ ID NO: 63) or L73A (SEQ ID NO: 64), wherein the position of each substitution is calculated with respect to the first amino acid residue at the N-terminus of SEQ ID NO: 2.

In certain embodiments, the amino acid residue at the N-terminus of the polypeptide according to the invention is acetylated. In some embodiments, the G amino acid residue at the N-terminus of the polypeptide of sequence SEQ ID NO: 6 to SEQ ID NO: 28 is acetylated.

In certain embodiments, the amino acid residue at the C-terminus of the polypeptide according to the invention is amidated. In some embodiments, the P amino acid residue at the C-terminus of the polypeptide of sequence SEQ IS NO: 6 to SEQ ID NO: 51 is amidated.

In some embodiments, the amino acid sequence of the polypeptide is not SEQ ID NO: 87. In some embodiments, the amino acid sequence of the polypeptide does not comprise SEQ ID NO: 87. In some embodiments, the polypeptide does not consist of an amino acid sequence having 100, 99, 98, 97, 96, 95, 90, 85, 80 or 75% identity with SEQ ID NO: 87. In some embodiments, the polypeptide does not comprise an amino acid sequence having 100, 99, 98, 97, 96, 95, 90, 85, 80 or 75% identity with SEQ ID NO: 87.

In some embodiments, the polypeptide is not a detection reagent for one organ-specific protein.

In certain embodiments, the amino acid residue at the C-terminus of the polypeptide according to the invention is further N-alkyl amidated or N-aryl amidated.

In some embodiments, the —OH group of the free —COOH group of the last amino acid residue at the C-terminus of the polypeptide is replaced by a group selected from an —O-alkyl group, an —O-aryl group, a —NH₂ group, a —N-alkyl amine group, a —N-aryl amine group or a —N-alkyl/aryl group.

Non-limitative examples of suitable alkyl groups include an alkyl in C₁-C₁₂.

Non-limitative examples of aryl groups include a phenyl, a tolyl, a xylyl or a naphtyl group, which may be substituted by one or more atom(s) or group(s) selected from O, N, —OH, —NH₂, a C₁-C₁₂ alkyl group, and a halogen (F, Cl, Br, I). Non-limitative examples of —N-alkyl amine groups include —NR¹R² groups, wherein R¹ and R² represent H or a C₁-C₁₂ alkyl group. Non-limitative examples of a —N-aryl amine group include —NHR³, wherein R³ represents a phenyl, a tolyl, a xylyl or a naphtyl group, which may be substituted by one or more atom(s) or group(s) from O, N, —OH, —NH₂, a C₁-C₁₂ alkyl group, and a halogen (F, Cl, Br, I). Non-limitative examples of —N-alkyl/aryl group include —NR⁴R, wherein R⁴ represents an alkyl in C₁-C₁₂ and wherein R⁵ represents phenyl, a tolyl, a xylyl or a naphtyl group, which may be substituted by one or more atom(s) or group(s) from O, N, —OH, —NH₂, a C₁-C₁₂ alkyl group, and a halogen (F, Cl, Br, I).

In practice, the replacement of the —OH group of the free —COOH group may be performed accordingly to any suitable method known from the state in the art, or a method adapted therefrom.

In some embodiment, said lactate dehydrogenase subunit is lactate dehydrogenase H (LDH-H) subunit (also referred to as LDH-1).

In some embodiments, said lactate dehydrogenase subunit is lactate dehydrogenase M (LDH-M) subunit (also referred to as LDH-5).

In a particular embodiment, the polypeptide of the invention is capable of preventing the formation of a functional tetramer of LDH-H subunits (corresponding to isoform LDH-1) by interacting with the amino acid residues L166, A169, R170, P183, K246, W251, A252 and L255 of a full length LDH-H subunit of sequence SEQ ID NO: 2.

The present invention also relates to derivatives of a polypeptide as defined herein.

Indeed, the present invention also encompasses any polypeptide differing from a polypeptide specifically disclosed herein, e.g., a polypeptide of amino acid sequence SEQ ID NO: 5, by one or more substitutions, deletions, additions and/or insertions. Such derivatives may be naturally occurring or may be synthetically generated, for example, by modifying one or more of the above polypeptide sequences of the invention and evaluating one or more inhibiting activities of the polypeptide of the invention and/or using any of a number of techniques well known in the art.

Modifications may be made in the structure of the polypeptides of the present invention and still obtain a functional molecule that encodes a derivative polypeptide with desirable characteristics. When it is desired to alter the amino acid sequence of a polypeptide according to the invention to create an equivalent, or even an improved, variant or portion, one skilled in the art will typically change one or more of the codons of the encoding nucleic acid (e.g., DNA) sequence.

For example, certain amino acid residues may be substituted by other amino acid residues in a protein structure without appreciable loss of its ability to bind other polypeptides (e.g., polypeptide LDH-Htr of sequence SEQ ID NO: 4). Since it is the binding capacity and nature of a protein that defines that protein's biological functional activity, certain amino acid sequence substitutions can be made in a protein sequence, and, of course, its underlying DNA coding sequence, and nevertheless obtain a protein with similar properties.

It is thus contemplated that various changes may be made in the polypeptide sequences of the present invention, or in the corresponding nucleic acid sequences (e.g., DNA sequences) that encode said polypeptides without appreciable loss of their inhibiting activity. In many instances, a variant of a polypeptide according to the invention will contain one or more conservative substitutions. A “conservative substitution” is one in which an amino acid residue is substituted for another amino acid residue that has similar properties, such that one skilled in the art of peptide chemistry would expect the secondary structure and hydropathic nature of the polypeptide to be substantially unchanged.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: amino acid residues R and K; amino acid residues D and E; amino acid residues S and T; amino acid residues Q and N; and amino acid residues A, V. L and I.

Amino acid substitutions may further be made on the basis of similarity in polarity, charge, solubility, hydrophobicity, hydrophilicity and/or the amphipathic nature of the amino acid residues. For example, negatively charged amino acid residues include amino acid residues D and E; positively charged amino acid residues include amino acid residues K and R; and amino acid residues with uncharged polar head groups having similar hydrophilicity values include amino acid residues A, L, I and V; amino acid residues G and A; amino acid residues N and Q; and amino acid residues S, T, F and Y. Other groups of amino acid residues that may represent conservative changes include: (1) amino acid residues A, P, G, E, D, Q, N, S, T; (2) amino acid residues C, S, Y, T; (3) amino acid residues V, I, L, M, A, F; (4) amino acid residues K, R, H; and (5) amino acid residues F, Y, W, H.

A derivative of the polypeptide according to the invention may also, or alternatively, contain nonconservative changes. In another embodiment, a derivative differs from a polypeptide sequence by substitution, deletion or addition of five amino acid residues or fewer. Derivatives may also (or alternatively) be modified by, e.g., the deletion or addition of amino acid residues that have minimal influence on the inhibitory capacity of the polypeptide according to the invention.

In another particular embodiment, the polypeptide of the invention comprises whole or part of the tetramerization domain of a lactate dehydrogenase subunit, and more specifically of lactate dehydrogenase M (LDH-M) or lactate dehydrogenase H (LDH-H) subunit.

In some embodiments, the polypeptide according to the invention comprises at least 16 amino acid residues. Within, the scope of the invention, the expression “at least 16 amino acid residues” encompasses 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more amino acid residues.

In certain embodiments, the polypeptide of the invention comprises from 16 to 200 amino acid residues, preferably from 16 to 150 amino acid residues, more preferably from 16 to 125 amino acid residues. In some embodiment, the polypeptide of the invention comprises from 16 to 100 amino acid residues, preferably from 16 to 75 amino acid residues, from 16 to 50 amino acid residues, or from 16 to 40 amino acid residues. In certain embodiment, the polypeptide of the invention comprises from 16 to 30 amino acid residues, from 16 to 22 amino acid residues, or from 16 to 20 amino acid residues.

In certain embodiments, the polypeptide of the invention comprises from 22 to 200 amino acid residues, preferably from 22 to 150 amino acid residues, more preferably from 22 to 125 amino acid residues. In some embodiment, the polypeptide of the invention comprises from 22 to 100 amino acid residues, preferably from 22 to 75 amino acid residues, from 22 to 50 amino acid residues, or from 22 to 40 amino acid residues. In certain embodiment, the polypeptide of the invention comprises from 22 to 30 amino acid residues, or from 22 to 25 amino acid residues.

In one embodiment, the polypeptide of the invention comprises at most 200, 190, 180, 170, 160, 150, 140, 130, 120, 110, 90, 80, 70, 60, 50, 40, 30 amino acid residues. In a particular embodiment, the polypeptide of the invention comprises at most 22 amino acid residues, preferably at most 16 amino acid residues.

In one embodiment, the polypeptide of the invention comprises at most 332 or 334 amino acid residues. In one embodiment, the polypeptide of the invention comprises from 16 to 332 amino acid residues. In one embodiment, the polypeptide of the invention comprises from 16 to 334 amino acid residues.

In one embodiment, the polypeptide of the invention comprises at most 312 or 314 amino acid residues. In one embodiment, the polypeptide of the invention comprises less than 312 or 314 amino acid residues.

In some embodiments, the amino acid sequence of the polypeptide according to the invention is not SEQ ID NO: 1, SEQ ID NO: 2, SEQ ID NO: 3 or SEQ ID NO: 4.

In certain embodiments, the polypeptide according to the invention further comprises at least one additional amino acid sequence, hereinafter referred to as a “tag polypeptide”.

As used herein, the term “tag polypeptide” refers to a polypeptide allowing the polypeptide of the invention either to be specifically labelled with an epitope for being detected of purified, or to be targeted to specific cells, a specific tissue or a specific organ, i.e., to a specific body location of the subject. In said embodiment, said polypeptide further comprises at least one tag polypeptide.

Further, in a particular embodiment, the tag polypeptide further allows the polypeptide of the invention to be targeted in the cytoplasm, in the nucleus or in the organelles of target cells, and more preferably of cancer cells.

In a particular embodiment of the invention, the said tag polypeptide is short enough such that it does not interfere with the inhibitory activity of the polypeptide of the invention. Illustratively, suitable tag polypeptides generally have at least six amino acid residues, preferably between about 8 to about 50 amino acid residues, and more preferably, between about 10 to about 20 amino acid residues.

A tag polypeptide for use in the present invention may be such that it provides an epitope to which an anti-tag antibody can selectively bind or it enables the polypeptide of the invention to be readily purified by affinity purification using an anti-tag antibody or another type of affinity matrix that binds to the epitope tag.

Various tag polypeptides are well known in the art. Examples include poly-histidine (poly-his) or poly-histidine-glycine (poly-his-gly) tags; the flu HA tag polypeptide, the c-myc tag, the Herpes Simplex virus glycoprotein D (gD) tag, the Flag-peptide; the KT3 epitope peptide; an alpha-tubulin epitope peptide; and the T7 gene 10 protein peptide tag.

The polypeptide according to the invention may also be modified so that it can be more easily detected, e.g., by biotinylation or by incorporation of any detectable label known in the art such as radiolabels, fluorescent labels or enzymatic labels. In a particular embodiment, the polypeptide of the invention may thus further comprise any amino acid sequence allowing the said polypeptide to be purified or detected more easily (e.g., a His-Tag, a Biotin tag or a Streptavidin tag).

In a particular embodiment, the polypeptide according to the invention may thus further comprise at least one tag polypeptide consisting in a cell-penetrating peptide (CPPs), also known as protein transduction domain, that facilitates entry into cells. As is well known in the art, cell-penetrating peptides are generally short peptides of up to 30 amino acid residues having a net positive charge and act in a receptor-independent and energy-independent manner.

Thus, the polypeptide according to the invention may comprise one or more cell-penetrating peptides. If so, the cell-penetrating peptide may be cleavable inside a cell. Examples of CPPs include those selected in the group consisting of hydrophilic and amphipathic CPPs. Hydrophilic CPPs are peptides composed mainly by hydrophilic amino acids usually rich in amino acid residues R and K.

Non-limitative examples of hydrophilic CPPs include:

Antennapedia Penetratin,
(RQIKWFQNRRMKWKK, SEQ ID NO: 68)
TAT,
(YGRKKRRQRRR, SEQ ID NO: 69)
SynB1,
(RGGRLSYSRRRFSTSTGR, SEQ ID NO: 70)
SynB3,
(RRLSYSRRRF, SEQ ID NO: 71)
PTD-4,
(PIRRRKKLRRLK, SEQ ID NO: 72)
PTD-5,
(RRQRRTSKLMKR, SEQ ID NO: 73)
FHV Coat-(35-49),
(RRRRNRTRRNRRRVR, SEQ ID NO: 74)
BMV Gag-(7-25),
(KMTRAQRRAAARRNRWTAR, SEQ ID NO: 75)
HTLV-II Rex-(4-16),
(TRRQRTRRARRNR, SEQ ID NO: 76)
D-Tat
(GRKKRRQRRRPPQ, SEQ ID NO: 77),
and
R9-Tat
(GRRRRRRRRRPPQ, SEQ ID NO: 78).

Amphipathic CPPs are peptides usually rich in amino acid residue K. Non-limitative examples of amphipathic CPPs include antimicrobial peptides, such as MAP or transportan:

Transportan,
(GWTLNSAGYLLGKINLKALAALAKKIL, SEQ ID NO: 79)
MAP,
(KLALKLALKLALALKLA, SEQ ID NO: 80)
SBP,
(MGLGLHLLVLAAALQGAWSQPKKKRKV, SEQ ID NO: 81)
FBP,
(GALFLGWLGAAGSTMGAWSQPKKKRKV, SEQ ID NO: 82)
MPG,
(GALFLGFLGAAGSTMGAWSQPKKKRKV, SEQ ID NO: 83)
MPG(ANLS),
(GALFLGFLGAAGSTMGAWSQPKSKRKV, SEQ ID NO: 84)
Pep-1,
(KETWWETWWTEWSQPKKKRKV, SEQ ID NO: 85)
and
Pep-2
(KETWFETWFTEWSQPKKKRKV, SEQ ID NO: 86).

The antennapedia-derived penetratin and the TAT peptide, or their derivatives, are in particular widely used tools for the delivery of cargo molecules such as peptides, proteins and oligonucleotides into cells (Fischer et al.; Cellular Delivery of Impermeable Effector Molecules in the Form of Conjugates with Peptides Capable of Mediating Membrane Translocation; Bioconjugate Chem. 2001, 12, 6, 825-841). In another embodiment, the polypeptide of the invention may also comprise a cell-penetrating peptide such as those disclosed in the patent applications WO 2011/157713 and WO 2011/157715 (Hoffmann La Roche®), or derivatives thereof.

In a particular embodiment of the invention, the polypeptide according to the invention is linked to the at least one cell-penetrating peptide (CPPs) by a linker. Within the meaning of the present invention, by “linker”, it is meant a single covalent bond or a moiety comprising series of stable covalent bonds, the moiety often incorporating 1-40 plural valent atoms selected from the group consisting of C, N, O, S and P, that covalently attach a coupling function or a bioactive group to the ligand of the invention. The number of plural valent atoms in a linker may be, for example, 0, 1, 2, 3, 4, 5, 6, 7, 8, 9, 10, 20, 25, or 30 or a larger number up to 40 or more. A linker may be linear or non-linear, and some linkers may have pendant side chains or pendant functional groups (or both).

The polypeptides of the invention may be prepared by methods well known to the skilled person in the art, such as culturing cells transformed or transfected with a vector containing a nucleic acid encoding the desired polypeptide or alternative methods, such as direct peptide synthesis using solid-phase techniques, or in vitro protein synthesis.

The invention also relates to a nucleic acid encoding a polypeptide according to the instant invention.

In some embodiments, the nucleic acid comprises a DNA nucleic acid sequence.

The instant disclosure also relates to a nucleic acid vector comprising at least one nucleic acid according to the invention.

Within the scope of the instant invention, the expression “at least one nucleic acid” is intended to include 1, 2, 3, 4, 5, 6, 7, 8, 9, 10, 15, 20, 25, 30, 35, 40, 45, 50 or more nucleic acids.

In some embodiment, the vector allows the controlled expression of said at least one polypeptide. As used herein, the expression “controlled expression” is intended to refer to an expression that is controlled in time and/or space. In other words, the controlled expression of the polypeptide according to the invention may occur in a specific location of the body, such as, e.g., a specific organ, and/or for a specific time period.

In certain embodiment, the vector is a viral vector, preferably selected in a group comprising an adenovirus, an adeno-associated virus (AAV), an alphavirus, a herpesvirus, a lentivirus, a non-integrative lentivirus, a retrovirus, vaccinia virus and a baculovirus.

In some embodiments, the polypeptide, the nucleic acid or the nucleic acid vector according to the invention may be comprised in a delivery particle, in particular, in combination with other natural or synthetic compounds, such as, e.g., lipids, protein, peptides, or polymers.

Within the scope of the invention said delivery particle is intended to provide, or “deliver”, the target cells, tissue or organ with the polypeptide, nucleic acid or nucleic acid vector according to the invention.

In some embodiment, the delivery particle may be in the form of a lipoplex, comprising cationic lipids; a lipid nano-emulsion; a solid lipid nanoparticle; a peptide-based particle; a polymer-based particle, in particular comprising natural and/or synthetic polymers; and a mixture thereof.

In some embodiment, a polymer-based particle may comprise a synthetic polymer, in particular, a polyethylene imine (PEI), a dendrimer, a poly (DL-Lactide) (PLA), a poly(DL-Lactide-co-glycoside) (PLGA), a polymethacrylate and a polyphosphoesters.

In some embodiment, the delivery particle further comprises at its surface one or more ligand(s) suitable for addressing the polypeptide, the nucleic acid or the nucleic acid vector to a target cell, tissue or organ.

Another aspect of the invention pertains to a pharmaceutical composition comprising (i) at least one polypeptide, at least one nucleic acid, or at least one nucleic acid vector according to the invention and (ii) at least one pharmaceutically acceptable vehicle.

In some aspects, the invention relates to a pharmaceutical composition comprising (i) at least one polypeptide according to the invention, and (ii) at least one pharmaceutically acceptable vehicle.

In some embodiments, the pharmaceutically acceptable vehicle is selected in a group comprising or consisting of a solvent, a diluent, a carrier, an excipient, a dispersion medium, a coating, an antibacterial agent, an antifungal agent, an isotonic agent, an absorption delaying agent and any combinations thereof. The carrier, diluent, solvent or excipient must be “acceptable” in the sense of being compatible with the polypeptide, or derivative thereof, and not be deleterious upon being administered to an individual. Typically, the vehicle does not produce an adverse, allergic or other untoward reaction when administered to an individual, preferably a human individual.

For the particular purpose of human administration, the pharmaceutical compositions should meet sterility, pyrogenicity, general safety and purity standards as required by regulatory offices, such as, for example, the Food and Drugs Administration (FDA) Office or the European Medicines Agency (EMA).

In some embodiments, the carrier may be water or saline (e.g., physiological saline), which will be sterile and pyrogen free. Suitable excipients include mannitol, dextrose, lactose, starch, magnesium stearate, sodium saccharine, cellulose, magnesium carbonate, and the like.

Acceptable carriers, solvents, diluents and excipients for therapeutic use are well known in the pharmaceutical art, and are described, for example, in Remington's Pharmaceutical Sciences, Mack Publishing Co. (A. R. Gennaro ed. 1985). The choice of a suitable pharmaceutical carrier, solvent, excipient or diluent can be made with regard to the intended route of administration and standard pharmaceutical practice. The pharmaceutical compositions may comprise as, or in addition to, the carrier, excipient, solvent or diluent any suitable binder, lubricant, suspending agent, coating agent, or solubilizing agent. Preservatives, stabilizers, dyes and even flavoring agents may be provided in the pharmaceutical composition.

The formulations may conveniently be presented in unit dosage form and may be prepared by any of the methods and the good practices well known in the art of pharmacy. Such methods include the step of bringing into association the polypeptide with the carrier which constitutes one or more accessory ingredients.

Formulations in accordance with the present invention that are suitable for oral administration may be presented as discrete units such as capsules, cachets or tablets, each containing a predetermined amount of the polypeptide according to the invention; as a powder or granules; as a solution or a suspension in an aqueous liquid or a non-aqueous liquid; or as an oil-in-water liquid emulsion or a water-in-oil liquid emulsion. The polypeptide of the invention may also be presented as a bolus, electuary or paste.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets. The formulations for use in the present invention may further include other agents conventional in the art having regard to the type of formulation in question, for example those suitable for oral administration may include flavoring agents.

The present invention further concerns a medicament comprising at least one polypeptide, nucleic acid, vector or delivery particle according to the invention.

A further aspect of the invention kit comprising (i) at least one polypeptide, at least one nucleic acid, at least one nucleic acid vector, or at least one pharmaceutical composition according to the invention, and (ii) at least a means to administer the polypeptide, the nucleic acid, the nucleic acid vector, or the pharmaceutical composition.

In certain embodiments, the means to administer the polypeptide, the nucleic acid, the nucleic acid vector, or the pharmaceutical composition according to the invention may include a syringe, a trocar, a catheter, a cup, a spatula, and the likes.

In some embodiments, the kit further comprises an anticancer agent.

Anticancer agents are known from the state of the art. Non-limitative examples of anticancer agents include acalabrutinib, alectinib, alemtuzumab, anastrozole, avapritinib, avelumab, belinostat, bevacizumab, bleomycin, blinatumomab, bosutinib, brigatinib, carboplatin, carmustine, cetuximab, chlorambucil, cisplatin copanlisib, cytarabine, daunorubicin, decitabine, dexamethasone, docetaxel, doxorubicin, encorafenib, erdafitinib, etoposide, everolimus, exemestane, fludarabine, 5-fluorouracil, gemcitabine, ifosfamide, imatinib Mesylate, leuprolide, lomustine, mechlorethamine, melphalan, methotrexate, mitomycin, nelarabine, paclitaxel, pamidronate, panobinostat, pralatrexate, prednisolone, ofatumumab, rituximab, temozolomide, topotecan, tositumomab, trastuzumab, vandetanib, vincristine, vorinostat, zanubrutinib, and the likes.

In certain embodiments, the anticancer agent is to be administered in combination with, concomitantly or sequentially, the polypeptide, the nucleic acid, the nucleic acid vector, or the pharmaceutical composition according to the invention.

One aspect of the invention relates to a polypeptide, a nucleic acid, a nucleic acid vector, or a pharmaceutical composition according to the instant invention, for use as a medicament.

In some further aspect, the invention also relates to the use of a polypeptide, nucleic acid, nucleic acid vector, or pharmaceutical composition according to the invention for the manufacture or the preparation of a medicament.

One aspect of the invention pertains to a polypeptide, nucleic acid, nucleic acid vector, or pharmaceutical composition, for use in preventing and/or treating cancer.

In some embodiments, the cancer to be prevented/treated is characterized by metabolic reprogramming. In some embodiments, the cancer to be prevented/treated is characterized in that cancer cells have elevated glycolytic flux. In some embodiments, the cancer to be prevented/treated is characterized in that cancer cells have elevated lactate production.

The invention also relates to a method for preventing and/or treating cancer in an individual in need thereof, comprising at least the step of administering to the individual a therapeutically effective amount of a polypeptide, nucleic acid, nucleic acid vector, or pharmaceutical composition according to the instant invention.

The present invention also relates to a polypeptide, nucleic acid, nucleic acid vector, or pharmaceutical composition according to the invention, for use in inhibiting the expansion of cancer cells.

The present invention also relates to a method for inhibiting the expansion of cancer cells in an individual in need thereof, comprising at least the step of administering to the individual a therapeutically effective amount of polypeptide, nucleic acid, nucleic acid vector, or pharmaceutical composition according to the invention.

The present invention also concerns a polypeptide, nucleic acid, nucleic acid vector, or pharmaceutical composition according to the invention, for use in improving the overall survival of an individual having cancer.

The present invention also concerns a method for improving the overall survival of an individual having cancer, comprising at least the step of administering to the individual a therapeutically effective amount of polypeptide, nucleic acid, nucleic acid vector, or pharmaceutical composition according to the invention.

The present invention also concerns a polypeptide, nucleic acid, nucleic acid vector, or pharmaceutical composition according to the invention, for use in improving the prognosis of an individual having cancer.

The present invention also concerns a method for improving the prognostic of an individual having cancer, comprising at least the step of administering to the individual a therapeutically effective amount of polypeptide, nucleic acid, nucleic acid vector, or pharmaceutical composition according to the invention.

As used herein, the term “cancer” is intended to refer to all neoplastic cell growth and proliferation, whether malignant or benign, and all pre-cancerous and cancerous cells and tissues.

Within the scope of the invention, the terms “cancer” and “cancerous” are intended to refer to, or to describe, the physiological condition in mammals that is typically characterized by unregulated cell growth or proliferation. Examples of cancer include, but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulvar cancer, thyroid cancer, hepatic carcinoma and various types of head and neck cancer.

In another aspect, the present invention further concerns a polypeptide, nucleic acid, nucleic acid vector, or pharmaceutical composition according to the invention, for use in preventing and/or treating a cancer involving oxidative cancerous cells and/or glycolytic cancerous cells.

One further aspect of the invention pertains to a polypeptide, nucleic acid, nucleic acid vector, or pharmaceutical composition, for use in preventing and/or treating cancer in an individual in need thereof.

The present invention also relates to a polypeptide, nucleic acid, nucleic acid vector, or pharmaceutical composition according to the invention, for use in inhibiting the expansion of cancer cells in an individual in need thereof.

As used herein, “individual”, it is meant to refer to a mammal or non-mammal animal, and preferably a human.

In some embodiments, a non-human animal may be selected in a group of valuable economic or pet animals comprising a dog, a cat, a rat, a mouse, a monkey, cattle, a sheep, a goat, a pig and a horse.

In some embodiments, the “individual in need thereof” has been diagnosed as having cancer and/or metastasis. In certain embodiments, the individual is susceptible to develop cancer and/or metastasis. In some embodiments, the “individual in need thereof” is at risk of developing cancer and/or metastasis. In certain embodiments, the “individual in need thereof” has already been treated for cancer and/or metastasis.

In some embodiments, the individual to be treated with the polypeptide, nucleic acid, nucleic acid vector, or pharmaceutical composition according to the invention, may further be administered a further anticancer agent Illustratively, when preventing or treating breast cancer, the further therapeutic agent may be an agent known to prevent or treat breast cancer. Similarly, when preventing or treating uterine cancer, the further therapeutic agent may be an agent known to prevent or treat uterine cancer.

Illustratively, the further anticancer agent may be any anticancer agent known in the art. Examples of further anticancer therapeutic agent include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, NJ), and doxetaxel (TaxotereDD, Rhone-Poulenc Rorer, Antony, France), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), melphalan and other related nitrogen mustards. Also included in this definition are hormonal agents that act to regulate or inhibit hormone action on tumors such as tamoxifen and onapristone.

It is appreciated that the further anticancer agent may be administered at the same time as the polypeptide of the invention (i.e. simultaneous administration optionally in a co-formulation) or at a different time to the polypeptide (i.e. sequential administration where the further therapeutic agent is administered before or after the polypeptide is administered). The further anticancer agent may be administered in the same way as the polypeptide of the invention, or by using the usual administrative routes for that further anticancer agent.

The present invention further relates to a polypeptide, nucleic acid, nucleic acid vector, or pharmaceutical composition according to the invention, for use in blocking basal autophagy in an individual in need thereof.

The present invention further relates to a method for blocking basal autophagy in an individual in need thereof, comprising at least the step of administering to the individual a therapeutically effective amount of polypeptide, nucleic acid, nucleic acid vector, or pharmaceutical composition according to the invention.

As used herein, the term “basal autophagy” is intended to refer to the macroautophagic activity during cellular growth in normal medium containing amino acids and serum, which appears to be highly active in many cell types and in animal tissues.

Some aspect of the invention relates to the use of a polypeptide, nucleic acid, nucleic acid vector, or pharmaceutical composition according to the instant invention, for inhibiting the tetramerization of the lactate dehydrogenase subunits.

In some embodiments, the lactate dehydrogenase subunits are LDH-1 subunits and/or LDH-5 subunits.

In practice, LDH-1 subunits are referred to as LDH-H subunits, as isoform LDH-1 consists of 4 LDH-H subunits; whereas LDH-5 subunits are referred to as LDH-M subunits, as isoform LDH-5 consists of 4 LDH-M subunits.

In some embodiments, the lactate dehydrogenase subunits are LDH-1 subunits.

In some embodiments, the polypeptide, nucleic acid, nucleic acid vector, or pharmaceutical composition according to the present invention is to be administered orally, parenterally, topically, by inhalation spray, rectally, nasally, buccally, vaginally or via an implanted reservoir. The term administration used herein includes subcutaneous, intravenous, intramuscular, intraocular, intra-articular, intra-synovial, intrasternal, intrathecal, intrahepatic, intralesional and intracranial injection or infusion techniques.

In a preferred embodiment, the polypeptide, nucleic acid, nucleic acid vector, or pharmaceutical composition of the present invention is to be administered parenterally, subcutaneously, intravenously, or via an implanted reservoir.

In some embodiments, the polypeptide, nucleic acid, nucleic acid vector, or pharmaceutical composition of the invention is in a form adapted for injection, such as, for example, for intraocular, intramuscular, subcutaneous, intradermal, transdermal or intravenous injection or infusion.

Examples of forms adapted for injection include, but are not limited to, solutions, such as, for example, sterile aqueous solutions, dispersions, emulsions, suspensions, solid forms suitable for using to prepare solutions or suspensions upon the addition of a liquid prior to use, such as, for example, powder, liposomal forms and the like.

The treatment may consist of a single dose or a plurality of doses over a period of time. The polypeptide, nucleic acid, nucleic acid vector, or pharmaceutical composition according to the invention may be formulated in a sustained release formulation so as to provide sustained release over a prolonged period of time such as over at least 2 or 4 or 6 or 8 weeks. Preferably, the sustained release is provided over at least 4 weeks.

In certain embodiments, the effective amount of the polypeptide, nucleic acid, nucleic acid vector, or pharmaceutical composition to be administered may depend upon a variety of parameters, including the material selected for administration, whether the administration is in single or multiple doses, and the subject's parameters including age, physical conditions, size, weight, gender, and the severity of the cancer to be treated.

In some embodiments, the polypeptide according to the invention is administered to the subject in need thereof in a therapeutically effective amount.

By “therapeutically effective amount”, it is meant a level or amount of polypeptide, or of a pharmaceutical composition, that is necessary and sufficient for slowing down or stopping the progression, aggravation, or deterioration of one or more symptoms of cancer; or alleviating the symptoms of cancer; or curing cancer, without causing significant negative or adverse side effects to the individual.

In certain embodiments, an effective amount of the polypeptide according to the invention may range from about 0.001 mg to about 3,000 mg, per dosage unit, preferably from about 0.05 mg to about 1,000 mg, per dosage unit.

Within the scope of the instant invention, from about 0.001 mg to about 3,000 mg includes, from about 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,100 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg and 3,000 mg per dosage unit.

In certain embodiments, the polypeptide according to the invention is to be administered at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day.

In some embodiments, an effective amount of the nucleic acid or nucleic acid vector to be administered may range from about 1×10⁵ to about 1×10¹⁵ copies per dosage unit.

Within the scope of the instant invention, from about 1×10⁵ to about 1×10¹⁵ copies includes 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10, 2×10⁸, 3×10⁸, 4×10⁸, 5×10⁸, 6×10⁸, 7×10⁸, 8×10⁸, 9×10⁹, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁵ and 1×10¹⁵ copies, per dosage unit.

Sequences Used Herein

TABLE 1
Sequences used herein

| SEQ | | |
|---|---|---|
| ID | | |
| NO: | Name | Sequences |
| 1 | hLDH-M | MATLKDQLIYNLLKEEQTPQNKITVVGVGAVGMACAI |
| | subunit (also | SILMKDLADELALVDVIEDKLKGEMMDLQHGSLFLRT |
| | referred to | PKIVSGKDYNVTANSKLVIITAGARQQEGESRLNLVQR |
| | LDH-5) | NVNIFKFIIPNVVKYSPNCKLLIVSNPVDILTYVAWKISG |
| | | FPKNRVIGSGCNLDSARFRYLMGERLGVHPLSCHGWV |
| | | LGEHGDSSVPVWSGMNVAGVSLKTLHPDLGTDKDKE |
| | | QWKEVHKQVVESAYEVIKLKGYTSWAIGLSVADLAES |
| | | IMKNLRRVHPVSTMIKGLYGIKDDVFLSVPCILGQNGIS |
| | | DLVKVTLTSEEEARLKKSADTLWGIQKELQF |
| 2 | hLDH-H | MATLKEKLIAPVAEEEATVPNNKITVVGVGQVGMACA |
| | subunit (also | ISILGKSLADELALVDVLEDKLKGEMMDLQHGSLFLQT |
| | referred to | PKIVADKDYSVTANSKIVVVTAGVRQQEGESRLNLVQ |
| | LDH-1) | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 3 | hLDH-Mtr | NKITVVGVGAVGMACAISILMKDLADELALVDVIEDK |
| | | LKGEMMDLQHGSLFLRTPKIVSGKDYNVTANSKL VIIT |
| | | AGARQQEGESRLNLVQRNVNIFKFIIPNVVKYSPNCKL |
| | | LIVSNPVDILTYVAWKISGFPKNRVIGSGCNLDSARFRY |
| | | LMGERLGVHPLSCHGWVLGEHGDSSVPVWSGMNVAG |
| | | VSLKTLHPDLGTDKDKEQWKEVHKQVVESAYEVIKLK |
| | | GYTSWAIGLSVADLAESIMKNLRRVHPVSTMIKGLYGI |
| | | KDDVFLSVPCILGQNGISDLVKVTLTSEEEARLKKSADT |
| | | LWGIQKELQF |
| 4 | hLDH-Htr | NNKITVVGVGQVGMACAISILGKSLADELALVDVLED |
| | | KLKGEMMDLQHGSLFLQTPKIVADKDYSVTANSKIVV |
| | | VTAGVRQQEGESRLNLVQRNVNVFKFIIPQIVKYSPDCI |
| | | IIVVSNPVDILTYVTWKLSGLPKHRVIGSGCNLDSARFR |
| | | YLMAEKLGIHPSSCHGWILGEHGDSSVAVWSGVNVAG |
| | | VSLQELNPEMGTDNDSENWKEVHKMVVESAYEVIKL |
| | | KGYTNWAIGLSVADLIESMLKNLSRIHPVSTMVKGMY |
| | | GIENEVFLSLPCILNARGLTSVINQKLKDDEVAQLKKSA |
| | | DTLWDIQKDLKDL |
| 5 | GP-X | GX1MMX2LQHGSX3X4X5QTP |
| 6 | GP-16 | GEMMDLQHGSLFLQTP |
| 7 | GP-16/E62D | GDMMDLQHGSLFLQTP |
| 8 | GP-16/E62A | GAMMDLQHGSLFLQTP |
| 9 | GP-16/D65E | GEMMELQHGSLFLQTP |
| 10 | GP-16/D65A | GEMMALQHGSLFLQTP |
| 11 | GP-16/L71A | GEMMDLQHGSAFLQTP |
| 12 | GP-16/L71V | GEMMDLQHGSVFLQTP |
| 13 | GP-16/L71I | GEMMDLQHGSIFLQTP |
| 14 | GP-16/L71F | GEMMDLQHGSFFLQTP |
| 15 | GP-16/L71W | GEMMDLQHGSWFLQTP |
| 16 | GP-16/L71Y | GEMMDLQHGSYFLQTP |
| 17 | GP-16/F72A | GEMMDLQHGSLALQTP |
| 18 | GP-16/F72L | GEMMDLQHGSLLLQTP |
| 19 | GP-16/F72V | GEMMDLQHGSLVLQTP |
| 20 | GP-16/F72I | GEMMDLQHGSLILQTP |
| 21 | GP-16/F72W | GEMMDLQHGSLWLQTP |
| 22 | GP-16/F72Y | GEMMDLQHGSLYLQTP |
| 23 | GP-16/L73A | GEMMDLQHGSLFAQTP |
| 24 | GP-16/L73V | GEMMDLQHGSLFVQTP |
| 25 | GP-16/L73I | GEMMDLQHGSLFIQTP |
| 26 | GP-16/L73F | GEMMDLQHGSLFFQTP |
| 27 | GP-16/L73W | GEMMDLQHGSLFWQTP |
| 28 | GP-16/L73Y | GEMMDLQHGSLFYQTP |
| 29 | LP-22 | LEDKLKGEMMDLQHGSLFLQTP |
| 30 | LP-22/E62D | LEDKLKGDMMDLQHGSLFLQTP |
| 31 | LP-22/E62A | LEDKLKGAMMDLQHGSLFLQTP |
| 32 | LP-22/D65E | LEDKLKGEMMELQHGSLFLQTP |
| 33 | LP-22/D65A | LEDKLKGEMMALQHGSLFLQTP |
| 34 | LP-22/L71A | LEDKLKGEMMDLQHGSAFLQTP |
| 35 | LP-22/L71V | LEDKLKGEMMDLQHGSVFLQTP |
| 36 | LP-22/L71I | LEDKLKGEMMDLQHGSIFLQTP |
| 37 | LP-22/L71F | LEDKLKGEMMDLQHGSFFLQTP |
| 38 | LP-22/L71W | LEDKLKGEMMDLQHGSWFLQTP |
| 39 | LP-22/L71Y | LEDKLKGEMMDLQHGSYFLQTP |
| 40 | LP-22/F72A | LEDKLKGEMMDLQHGSLALQTP |
| 41 | LP-22/F72L | LEDKLKGEMMDLQHGSLLLQTP |
| 42 | LP-22/F72V | LEDKLKGEMMDLQHGSLVLQTP |
| 43 | LP-22/F72I | LEDKLKGEMMDLQHGSLILQTP |
| 44 | LP-22/F72W | LEDKLKGEMMDLQHGSLWLQTP |
| 45 | LP-22/F72Y | LEDKLKGEMMDLQHGSLYLQTP |
| 46 | LP-22/L73A | LEDKLKGEMMDLQHGSLFAQTP |
| 47 | LP-22/L73V | LEDKLKGEMMDLQHGSLFVQTP |
| 48 | LP-22/L73I | LEDKLKGEMMDLQHGSLFIQTP |
| 49 | LP-22/L73F | LEDKLKGEMMDLQHGSLFFQTP |
| 50 | LP-22/L73W | LEDKLKGEMMDLQHGSLFWQTP |
| 51 | LP-22/L73Y | LEDKLKGEMMDLQHGSLFYQTP |
| 52 | hLDH- | MATLKEKLIAPVAEEEATVPNNKITVVGVGQVGMACA |
| | 1/G61A | ISILGKSLADELALVDVLEDKLKAEMMDLQHGSLFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 53 | hLDH- | MATLKEKLIAPVAEEEATVPNNKITVVGVGQVGMACA |
| | 1/E62A | ISILGKSLADELALVDVLEDKLKGAMMDLQHGSLFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 54 | hLDH- | MATLKEKLIAPVAEEEATVPNNKITVVGVGQVGMACA |
| | 1/M63A | ISILGKSLADELALVDVLEDKLKGEAMDLQHGSLFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 55 | hLDH- | MATLKEKLIAPVAEEEATVPNNKITVVGVGQVGMACA |
| | 1/M64A | ISILGKSLADELALVDVLEDKLKGEMADLQHGSLFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 56 | hLDH- | MATLKEKLIAPVAEEEATVPNNKITVVGVGQVGMACA |
| | 1/D65A | ISILGKSLADELALVDVLEDKLKGEMMALQHGSLFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 57 | hLDH- | MATLKEKLIAPVAEEEATVPNNKITVVGVGQVGMACA |
| | 1/L66A | ISILGKSLADELALVDVLEDKLKGEMMDAQHGSLFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 58 | hLDH- | MATLKEKLIAPVAEEEATVPNNKITVVGVGQVGMACA |
| | 1/Q67A | ISILGKSLADELALVDVLEDKLKGEMMDLAHGSLFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 59 | hLDH- | MATLKEKLIAPVAEEEATVPNNKITVVGVGQVGMACA |
| | 1/H68A | ISILGKSLADELALVDVLEDKLKGEMMDLQAGSLFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 60 | hLDH- | MATLKEKLIAPVAEEEATVPNNKITVVGVGQVGMACA |
| | 1/G69A | ISILGKSLADELALVDVLEDKLKGEMMDLQHASLFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 61 | hLDH- | MATLKEKLIAPVAEEEATVPNNKITVVGVGQVGMACA |
| | 1/S70A | ISILGKSLADELALVDVLEDKLKGEMMDLQHGALFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 62 | hLDH- | MATLKEKLIAPVAEEEATVPNNKITVVGVGQVGMACA |
| | 1/L71A | ISILGKSLADELALVDVLEDKLKGEMMDLQHGSAFLQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 63 | hLDH- | MATLKEKLIAPVAEEEATVPNNKITVVGVGQVGMACA |
| | 1/F72A | ISILGKSLADELALVDVLEDKLKGEMMDLQHGSLALQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 64 | hLDH- | MATLKEKLIAPVAEEEATVPNNKITVVGVGQVGMACA |
| | 1/L73A | ISILGKSLADELALVDVLEDKLKGEMMDLQHGSLFAQT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 65 | hLDH- | MATLKEKLIAPVAEEEATVPNNKITVVGVGQVGMACA |
| | 1/Q74A | ISILGKSLADELALVDVLEDKLKGEMMDLQHGSLFLAT |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 66 | hLDH- | MATLKEKLIAPVAEEEATVPNNKITVVGVGQVGMACA |
| | 1/T75A | ISILGKSLADELALVDVLEDKLKGEMMDLQHGSLFLQA |
| | | PKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 67 | hLDH- | MATLKEKLIAPVAEEEATVPNNKITVVGVGQVGMACA |
| | 1/P76A | ISILGKSLADELALVDVLEDKLKGEMMDLQHGSLFLQT |
| | | AKIVADKDYSVTANSKIVVVTAGVRQQEGESRLNLVQ |
| | | RNVNVFKFIIPQIVKYSPDCIIIVVSNPVDILTYVTWKLS |
| | | GLPKHRVIGSGCNLDSARFRYLMAEKLGIHPSSCHGWI |
| | | LGEHGDSSVAVWSGVNVAGVSLQELNPEMGTDNDSE |
| | | NWKEVHKMVVESAYEVIKLKGYTNWAIGLSVADLIES |
| | | MLKNLSRIHPVSTMVKGMYGIENEVFLSLPCILNARGL |
| | | TSVINQKLKDDEVAQLKKSADTLWDIQKDLKDL |
| 68 | Antennapedia | RQIKWFQNRRMKWKK |
| | Penetratin | |
| | CCP | |
| 69 | TAT CCP | YGRKKRRQRRR |
| 70 | SynB1 CCP | RGGRLSYSRRRFSTSTGR |
| 71 | SynB3 CCP | RRLSYSRRRF |
| 72 | PTD-4 CCP | PIRRRKKLRRLK |
| 73 | PTD-5 CCP | RRQRRTSKLMKR |
| 74 | FHV Coat- | RRRRNRTRRNRRRVR |
| | (35-49) CCP | |
| 75 | BMV Gag- | KMTRAQRRAAARRNRWTAR |
| | (7-25) CCP | |
| 76 | HTLV-II | TRRQRTRRARRNR |
| | Rex-(4-16) | |
| | CCP | |
| 77 | D-Tat CCP | GRKKRRQRRRPPQ |
| 78 | R9-Tat CCP | GRRRRRRRRRPPQ |
| 79 | Transportan | GWTLNSAGYLLGKINLKALAALAKKIL |
| | CCP | |
| 80 | MAP CCP | KLALKLALKLALALKLA |
| 81 | SBP CCP | MGLGLHLLVLAAALQGAWSQPKKKRKV |
| 82 | FBP CCP | GALFLGWLGAAGSTMGAWSQPKKKRKV |
| 83 | MPG CCP | GALFLGFLGAAGSTMGAWSQPKKKRKV |
| 84 | MPG(ANLS) | GALFLGFLGAAGSTMGAWSQPKSKRKV |
| | CCP | |
| 85 | PEP-1 CCP | KETWWETWWTEWSQPKKKRKV |
| 86 | PEP-2 CCP | KETWFETWFTEWSQPKKKRKV |
| 87 | PEP-17 | GEMMDLQHGSLFLQTPK |

### BRIEF DESCRIPTION OF THE DRAWINGS

FIG. 1A-E is a set of schemes showing the interaction mapping of LDH-H tetrameric interface highlights two main clusters. A: X-ray crystallographic structure of LDH-1 (LDH-H4) as a dimer of dimers with the two dimers (subunits A, C and B, D). B: Model of dimeric LDH-Htr. C: Model of dimeric LDH-H interacting with a single LDH-H subunit used to highlight LDH tetrameric interface (PDB ID: 1I0Z). D: Mapping of the interaction between an LDH-H subunit C and LDH-1 tetrameric interface (dimer B-D) using the MOE software. The x-axis and y-axis represent the residue numbers of dimer B-D and subunit C, respectively. This mapping identifies two clusters of interaction, clusters A and B. E: Representation of the different domains of native LDH-1 (UniProt P07195). Residue numbers are scaled to FIG. 1D x-axis.

FIG. 2A-E is a set of graphs showing that LDH-Htr behaves as a weak tetramer. A: Overlay of size exclusion chromatograms of LDH-Htr and LDH-1 using a Superdex 200 10/300 GL column. B: Evaluation of LDH-Htr self-interaction using microscale thermophoresis (MST) at a 20 s “on time” (n=3) (Kd=1.25 μM [0.96 to 1.62 μM]). C: Evaluation by nanoscale differential scanning fluorimetry (NanoDSF) of the impact of LDH-Htr melting temperature depending on its subunit concentration (n=3). Tm1 and Tm2 refer to the two transitions observed for LDH-Htr denaturation pattern. D: NanoDSF profile of LDH-Htr at various concentrations (n=3). RFU: Relative fluorescent unit. E: Mass photometry of LDH-Htr with the calculated molecular weights of the complex in solution and their relative intensity indicated above the peaks (theoretical Mw of the dimer is 73.2 kDa).

FIG. 3A-D is a set of graphs showing that polypeptide LP-22 interacts at the LDH tetrameric interface, destabilizes tetrameric LDH and stabilizes dimeric LDH. A: WaterLOGSY spectra of the interaction of LP-22 (400 μM) with dimeric LDH-Htr (upper curve) and with tetrameric LDH-1 (down curve) at 15 μM. B: MST binding curves between polypeptide LP-22 and LDH-Htr. Binding curves were extracted from the MST traces at a 1.5 s MST on time (n=3). C: NanoDSF denaturation of dimeric LDH-Htr (15 μM) with (curve 2) and without (curve 1) polypeptide LP-22 (500 μM) (ΔTm=2.8° C. n=3). D: ΔTm (° C.) of tetrameric LDH-5 (300 nM) as a function of polypeptide LP-22 concentration (EC50=47 μM [32 to 68 μM], n=3).

FIG. 4A-D is a set of schemes and graphs showing that polypeptide LP22 N-terminal trimming leads to polypeptide GP-16 with a similar interaction profile. A: (Upper panel) Comparison of the difference between polypeptide LP-22 (up) and polypeptide GP-16 (bottom) WaterLOGSY and ¹H NMR spectra in the presence of 15 μM of LDH-Htr. Signals that appear in the ¹H spectra but not in WaterLOGSY correspond to non-interacting residues. Polypeptide LP-22 spectra highlight that some lysines, glutamates, aspartates and leucines residues do not interact with LDH-Htr. These non-interacting signals are no longer present on the polypeptide GP-16 spectra (down). (Lower panel) Amino acid sequence of polypeptide LP-22. Dark colored residues correspond to the residues that do not interact according to ΔG calculation and WaterLOGSY analysis. B: Calculation of polypeptide LP-22 residue contribution to the overall free energy of binding using the MOE software. C: MST binding curves between polypeptide GP-16 and LDH-Htr. Binding curves were extracted from the MST traces at a 1.5 s MST on time (n=3). D: Differences in melting temperature (ΔTm, ° C.) of tetrameric LDH-5 (300 nM) as a function of polypeptide GP-16 concentration (EC50=262 μM [142 to 383 μM] n=3).

FIG. 5A-D is a set of graphs showing that mutations of cluster B1 unravel key residues for LDH tetramerization. Mass photometry was performed for LDH-1 (A), and for LDH-H variants E62A (B), L71A (C) and F72A (D) with the experimental molecular weights of the complexes in solution and their relative intensities. Theoretical molecular weight of the tetramer=155 kDa; Theoretical molecular weight of the dimer=78 kDa.

FIG. 6A-D is a set of graphs showing the exploitation of orthogonal methods highlights the impact of key mutations on LDH-H tetrameric stability. A: Tryptophan fluorescence spectra of LDH-1 (curve 1) and of different LDH-H variants, LDH-HE62A (curve 2), LDH-HL71A (curve 3) and LDH-HF72A (curve 4) (1.3 μM). λexc=286 nm (n=6). B: NanoDSF profiles of LDH-Htr (curve 1) and LDH-HD65A (curve 2) (n=6). C: NanoDSF profiles of LDH-HL66A (curve 1) and LDH-HL73A (curve 2) (n=6). D: Fluorescence intensity of tetrameric LDH-HL66A (curve 1) and LDH-HL73A (curve 2) at 50 μg/mL (1.3 μM) upon addition of guanidinium·HCl (n=6).

FIG. 7A-C is a set of graphs showing the structural model of the interaction between cluster B1 hot spots and cluster B2. A: Interaction of the sequence corresponding to polypeptide GP-16 with cluster B2. The surface corresponds to the molecular surface of LDH-H cluster B2. B: Focus on the hydrophobic hot spot of cluster B1 with the interaction made by L71, F72 and L73 with cluster B2. C: Focus on the hydrophilic hot spot of cluster B1 with the interaction made by D65 and E62 with cluster B2. This representation was isolated from the LDH-1 crystallographic structure and further minimized using the MOE software (PDB ID: 1I0Z).

### Examples

The present invention is further illustrated by the following examples.

1. Materials and Methods

1.1—Chemicals and Peptides

All reagents were purchased from different chemical suppliers and used without further purification. Peptides were purchased from Genecust® (https://www.genecust.com). Structure conformity and purity grade (>95%) were assessed by analytical high-performance liquid chromatography (HPLC) analysis and mass spectrometry (MS). Polypeptide GP-16 was amidated and acetylated respectively at its C- and N-termini and polypeptide LP-22 was only amidated at his C-terminus.

1.2—Production and Purification of Human LDH Proteins

The hLDH-H nucleotidic sequences used to produce full-length, truncated and variant LDH-H proteins inserted in a pET-28a expression vector were ordered from Genecust®. NdeI and Bpu1102I restriction sites were used for sequence insertion and allowed for an N-terminal 6-His tag addition. Protein production and purification were performed following a previously described in Thabault et aL (Interrogating the Lactate Dehydrogenase Tetramerization Site Using (Stapled) Peptides. J. Med. Chem. 2020, 63 (9), 4628-4643). Recombinant plasmids were then transformed in host bacterium E. coli Rosetta (DE3). Transformants were cultured in lysogeny broth (LB) medium supplemented with 50 μg/mL kanamycin and 34 μg/mL chloramphenicol at 37° C. until an optical density of 0.6 was reached. LDH expression was induced by the addition of 1 mM isopropyl-β-D-1-thiogalactopyranoside (IPTG) at 20° C. for 20 h. Then, cells were collected by centrifugation at 5,000 rpm (rotor 11150, Sigma^{®}), 4° C. for 25 min. Pellets were suspended in a lysis buffer (Tris-HCl 50 mM pH 8.5, MgCl₂ 10 mM, NaCl 300 mM, imidazole 5 mM and glycerol 10%), and then disrupted by sonication, followed by centrifugation at 4° C., 10,000 rpm (rotor 12165-H, Sigma®) for 30 min. The insoluble fraction was discarded, and 1 μL of β-mercaptoethanol was added per mL of soluble fraction. Purification of recombinant proteins was performed using 1 mL His-trap FF-crude columns (GE Healthcare®) according to the manufacturer's instructions. Finally, protein concentrations were measured using the Bradford method with the Protein Assay Kit (Biorad®), and sample homogeneity was assessed by sodium dodecyl sulfate-polyacrylamide gel electrophoresis (SDS-PAGE) with Coomassie brilliant blue as a staining agent.

Amino acid residue positions of LDH-H variants are calculated with respect to the 1^{st} amino acid residue at the N-terminus of LDH-H (also referred herein to as LDH-1; SEQ ID NO: 2).

1.3—Nuclear Magnetic Resonance

Human LDH-H (full length and truncated)-6His proteins for ¹D NMR were expressed and purified from E. coli, as described above. All experiments were performed on an Ascend Avance 111600 MHz system equipped with a broadband cryoprobe (Bruker®) following a previously described in Thabault et al. (see above).

For WaterLOGSY NMR studies, samples were prepared in 10% D₂O containing 50 mM sodium phosphate buffer, pH 7.6, and 100 mM NaCl. The concentration of LDHs was ranging from 15 to 20 μM of monomer. Ligand binding was detected using a WaterLOGSY ephogsygpno.2 avance-version sequence with a 1 s mixing time. Water signal suppression was achieved using an excitation-sculpting scheme, and a 50 ms spinlock was used to suppress protein background signals. For polypeptide LP-22 (SEQ ID NO: 29) and polypeptide GP-16 (SEQ ID NO: 6) spectra experiment, 4096 scans were collected at 277K to yield a 16K points free induction decay (FID).

1.4—in Silico Evaluation

Calculation of the free binding energy and mapping of the interaction at LDH interface was performed using the Molecular Operating Environment (MOE) software (ChemComp®) with the LDH-1 crystallographic structure (PDB entry 1I0Z). Following a previously described procedure (Thabault et al.; see above), tetrameric LDH-1 was generated from the PDB crystallographic structure using the MOE bioassembly tool. The truncated dimeric version of LDH-1 (LDH-Htr, SEQ ID NO: 4) was generated from the tetrameric complex by removing LDH-H subunits B and D, as well as the N-terminal domain of subunits A and C. Minimization was performed before free energy calculation and interaction mapping.

1.5—Size Exclusion Experiments The samples were loaded onto an equilibrated Superdex 200 10/300 GL column, run at 0.75 mUmin by an ÄKTApure® system (GE Healthcare®) using 50 mM sodium phosphate, pH 7.6, 100 mM NaCl as the mobile phase buffer. Following previously described procedures (Thabault et al.; see above), LDH-Htr was diluted to 15 μM in the assay buffer. The final injection volume was 500 μL. Molecular weights were determined using the gel filtration standard (Biorad®) in the same assay buffer following the manufacturer's instructions.

1.6—Nano Differential Scanning Fluorimetry Experiments

NanoDSF was performed following previously described by Thabault et al. (see above).
a) Variant evaluation

Solutions of proteins (LDH-H, LDH-Htr, or variants), stored in a 50 mM sodium phosphate, 100 mM NaCl and 20% glycerol, pH 7.6, were evaluated on a Tycho NT.6 device (NanoTemper Technologies®) using concentrations ranging from to 65 μM. According to standard manufacturer's procedures, samples were poured into capillaries and heated up to 95° C. in 3 min, while following fluorescence emission at 330 and 350 nm. Melting temperatures were extracted from the derivative of the 350/330 nm fluorescence ratios upon increasing temperature.
b) Peptide evaluation

Solutions of proteins (LDH-1, LDH-5 or LDH-Htr) with peptides were evaluated on a Tycho NT.6 device (NanoTemper Technologies®). Evaluations were performed in a 50 mM sodium phosphate and 100 mM NaCl pH 7.6 buffer. According to standard manufacturer's procedures, samples were poured into capillaries and treated as above.

1.7—Microscale Thermophoresis

MST measurements were performed on a Nanotemper® Monolith NT.115 instrument (NanoTemper Technologies®) using Red-dye-NHS fluorescent labelling. LDH-Htr purified to homogeneity was labelled with the Monolith Red-dye-NHS 2^{nd} generation labelling dye (Nanotemper Technologies®), according to the supplied protocol. Measurements were performed in 50 mM sodium phosphate, pH 7.6, and 100 mM NaCl containing 0.01% Tween-20 in standard-treated capillaries (NanoTemper Technologies®). The final concentration of proteins in the assay was 100 nM. Ligands were titrated in 1:1 dilutions following manufacturer's recommendations. Experiments were performed in triplicates using 40% LED power, high MST power, Laser on time s and Laser off time 3 s. Poypeptides were evaluated for their thermophoretic pattern, and Kd's were extracted from raw data at a 1.5 s MST on time following the manufacturer's instructions.

1.8—Mass Photometry

Protein landing was recorded using a Refeyn OneMP (Refeyn Ltd., UK) mass photometry system by adding 1 μL of the protein stock solution (1 μM) directly into a 16 μL drop of filtered PBS solution. Movies were acquired for 60 s (6,000 frames) with the AcquireMP (Refeyn Ltd., v2.1.1) software using standard settings. Data were analyzed using default settings on DiscoverMP (Refeyn Ltd, v2.1.1). Contrast-to-mass (C2M) calibration was performed prior to the experiments using a mix of proteins with molecular weights of 66 kDa, 146 kDa, 480 kDa and 1048 kDa.

1.9—Spectrophotometric Experiments

All spectrophotometric experiments were performed with opaque 96-well plates using a Spectramax m2e spectrophotometer (Molecular Devices) following previously described in Thabault et al. (see above).

Intrinsic fluorescence assays: full tryptophan fluorescence spectra were recorded using an excitation wavelength of 286 nm and recording the emission spectra from 320 to 400 nm at room temperature. The raw fluorescence of each experiment was subtracted to a corresponding control experiment without the protein. Experiments were performed in a 50 mM sodium phosphate and 100 mM NaCl, pH 7.6, buffer. For dissociation in subunits, increasing amounts of guanidinium-HCl ranging from 0.3 M to 2 M were put in contact with the studied proteins (1.3 μM), and fluorescence spectra were recorded afterwards.

1.10—Statistics

All quantitative data are expressed as means t SEM. Error bars are sometimes smaller than symbols. n refers to the total number of replicates. Data were analyzed using the GraphPad Prism 7.0 software.

2. Results

2.1—in Silico Mapping of the LDH-1 Tetrameric Interface Identifies a New Cluster of Interactions

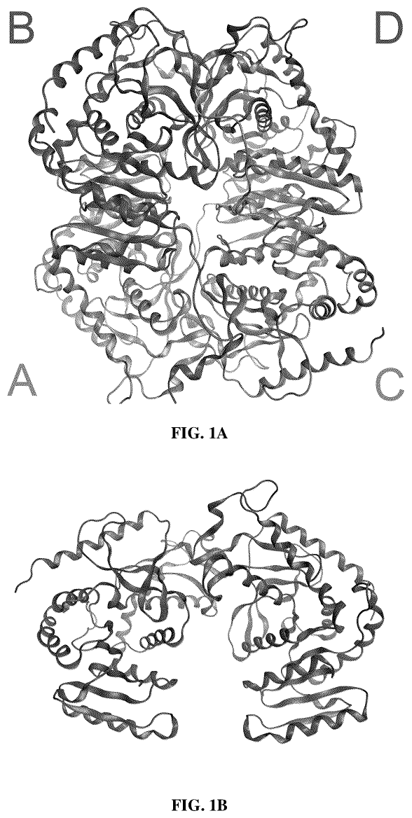

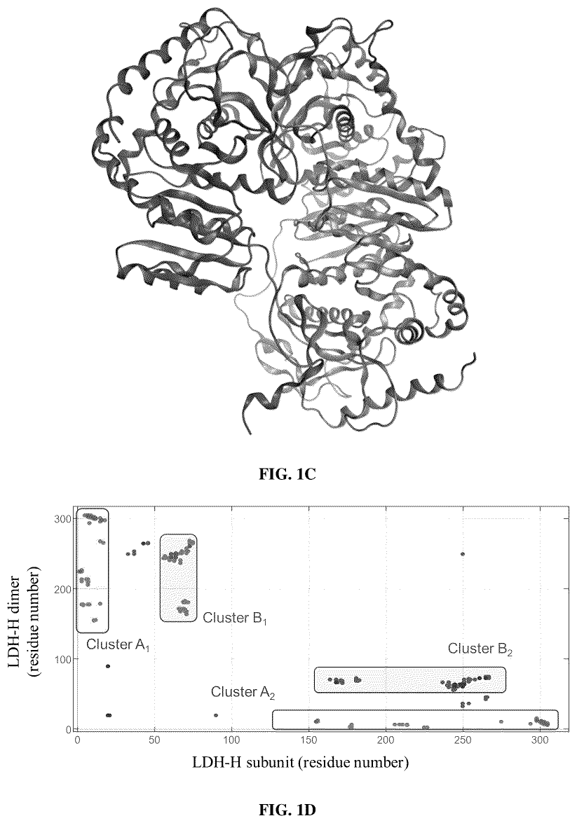

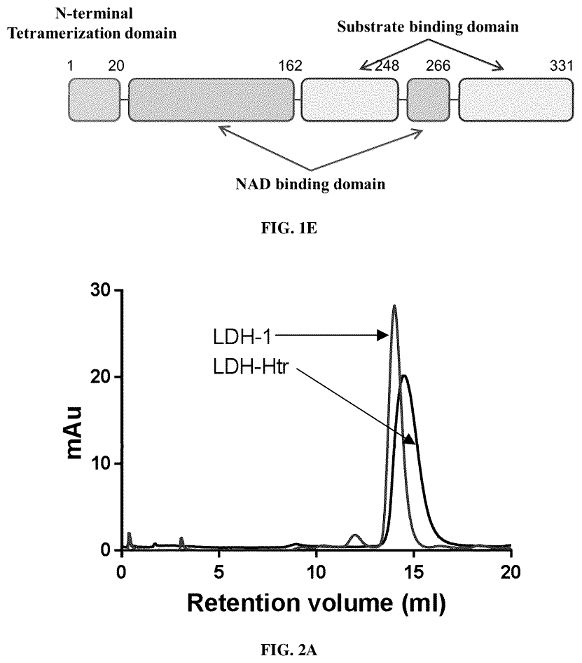

LDH quaternary state is a “dimer of dimers”. According to X-ray structures, three different subunit orientations could account for LDH dimeric conformation. In fact, LDH N-terminal domain truncation leads to dimers (LDH-Htr; SEQ ID NO: 4) (Thabault et al. (see above)). It was hypothesized that only the association of dimers A-C and B-D in a tetramer can explain the role of this N-terminal domain in the stabilization of the tetrameric state (FIG. 1A-C). Based on this hypothesis, the interactions made by one subunit with a LDH dimer (A-C or B-D) we first mapped using the Molecular Operating Environment (MOE) software. Mapping these contact points highlighted two clusters: A (A1 and A2) and B (B1 and B2) (FIG. 1D). Cluster A1 and A2 corresponded to the LDH N-terminal tetramerization domain (FIG. 1E) and to its related tetramerization site, respectively, which were previously in Thabault et al. (see above). Clusters B1 and B2 matched with a 22 amino acid α-helix and its interacting site, which were never reported before. Interestingly, the sequence corresponding to cluster B1 was highly conserved among vertebrates. Overall, Clusters A1, and B1 corresponded to continuous epitopes interacting with discontinuous oligomerization sites A2 and B2.

2.2—LDH-Htr Behaves as a Weak Tetramer Through Cluster B.

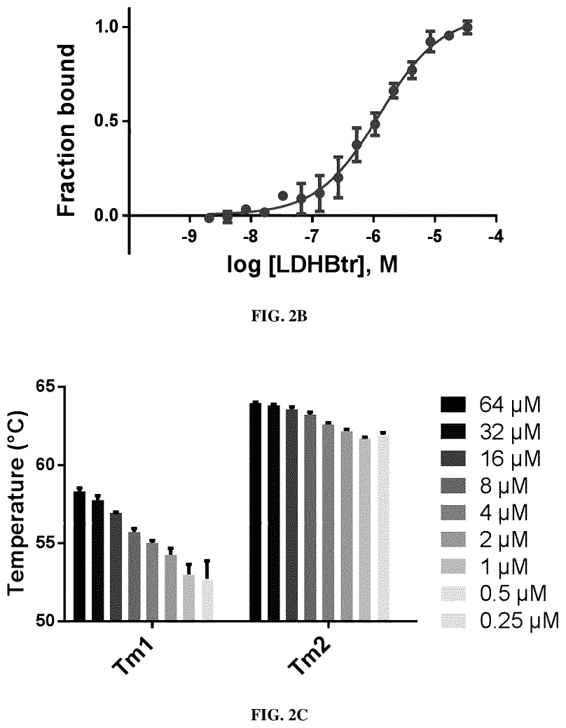

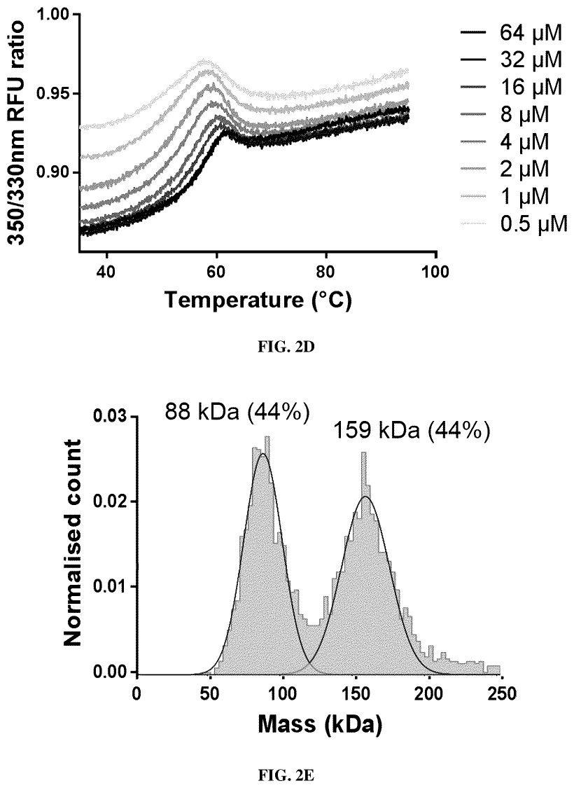

Next, it was aimed to confirm this interaction model and the anticipated symmetry axis of LDH dimers using the model of dimeric LDH (LDH-Htr; SEQ ID NO: 4) described in Thabault et aL (see above). According to the interaction map, LDH-Htr lacks cluster A1 but still possesses clusters A2, B1, and B2. It was thus reasoned that LDH-Htr might still be able to self-interact at high concentrations via cluster B. Comparison between LDH-Htr and LDH-1 elution profiles by size-exclusion chromatography (SEC) indeed suggested that LDH-Htr could be in an equilibrium between tetramers and dimers (FIG. 2A). Consistently, the evaluation of LDH-Htr self-interaction by Microscale thermophoresis (MST) revealed that the dimeric protein interacts with itself at high concentrations (FIG. 2B, Kd=1.25 μM [0.96 to 1.62 μM]). According to this model, this interaction can only be the result of LDH-Htr forming dimers of dimers through cluster B. Monitoring this interaction using MST hence provided valuable information on cluster B's overall potency. It was then evaluated whether LDH-Htr self-association could stabilize the protein complex, as the oligomerization of a protein often results in its stabilization. LDH-Htr denaturation profile was evaluated using nanoscale differential scanning fluorimetry (nanoDSF) and revealed that the protein exhibited a concentration-dependent destabilization (FIG. 2C) and conformational change (FIG. 2D). It was also evaluated LDH-Htr oligomeric state using mass photometry (MP). MP is a recent technique that allows for single-molecule detection and mass measurement in solution based on light scattering (Young et al.; Quantitative Mass Imaging of Single Biological Macromolecules. Science 2018, 360 (6387), 423-427). MP analysis of LDH-Htr revealed an equilibrium between dimers and tetramers in solution (FIG. 2E). Altogether, these results demonstrated that the truncation of the LDH N-terminal domain does not entirely prevent the protein from forming tetramers. This ability of the truncated dimers to interact and form weak tetramers validated the in silico model and provided valuable information about this new tetrameric interface.

2.3—Identification of Peptide Ligands of the LDH Tetrameric Interface

It was then set out to further characterize the continuous epitope B1 in order to identify peptides targeting the LDH tetrameric interface. As discussed above, cluster B1 corresponds to a 22 amino-acid peptide folding into a long and “kinked” α-helix ended by a short loop. It was thus decided to study the interaction between “cluster B1”-derived polypeptide (named LP-22, LEDKLKGEMMDLQHGSLFLQTP (SEQ ID NO: 29)) and the LDH-H tetrameric interface. To that end, a set of biophysical evaluation was performed using nuclear magnetic resonance (NMR) WaterLOGSY, MST and NanoDSF experiments.

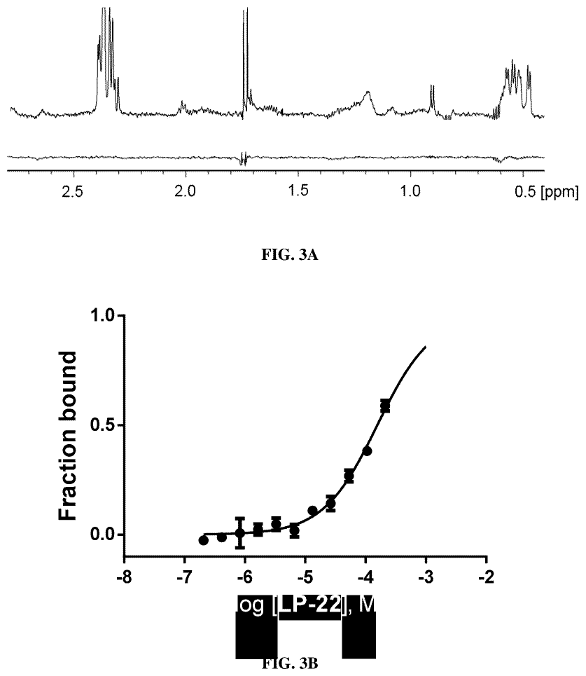

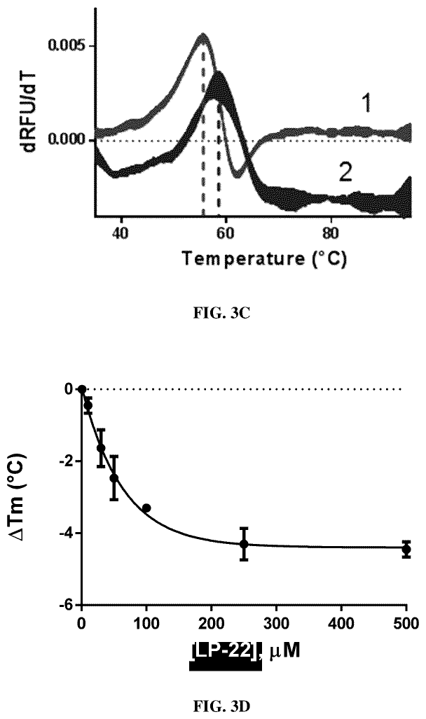

Strikingly, WaterLOGSY experiments showed that polypeptide LP-22 undergoes a saturation transfer with dimeric LDH-Htr, but not with the tetrameric LDH-1, thus demonstrating that it interacts at the LDH tetrameric interface (FIG. 3A). MST further validated this interaction with an estimated Kd of 156 μM with LDH-Htr (FIG. 3B). Thermal shift experiments using NanoDSF revealed a stabilization (ΔTm=2.8° C. at 500 μM) (FIG. 3C) of dimeric truncated LDH-Htr with polypeptide LP-22, consistent with an interaction occurring at the exposed oligomeric interface. On the opposite, polypeptide LP-22 destabilized tetrameric LDH in a concentration-dependent manner (FIG. 3D) with an EC50=47 μM [32 to 68 μM]. These results are coherent with the observation that ligands interacting at oligomeric interfaces often induce protein thermal destabilization. The LDH-5 tetramer was more destabilized than LDH-1, consistent with a difference in the stability of these two protein complexes that we previously reported in Thabault et al. (see above). Interestingly, a recent report of selective LDH-5 inhibitors disclosed an inhibitor interacting at LDH-5 tetrameric interface near cluster B (Friberg et aL; Structural Evidence for Isoform-Selective Allosteric Inhibition of Lactate Dehydrogenase A. ACS Omega 2020, 5 (22), 13034-13041). This inhibitor was highly selective towards LDH-5, which is coherent with the lower stability of LDH-5 tetrameric complex. Such selectivity profile is also consistent with the higher destabilizing effect that polypeptide LP-22 displays on LDH-5 compared to LDH-1. Consistently with our previous report on LDH tetramer disruptors (Thabault et aL (see above)), LDH destabilization was also dependent on protein concentration, as an increasing amount of subunit reverted the effect. Overall, these results consistently demonstrated that polypeptide LP-22 interacts at the LDH tetrameric interface and destabilizes the tetrameric enzyme.

2.4—Biophysical and Computational Experiments Identify Polypeptide LP-22 Essential Binding Region

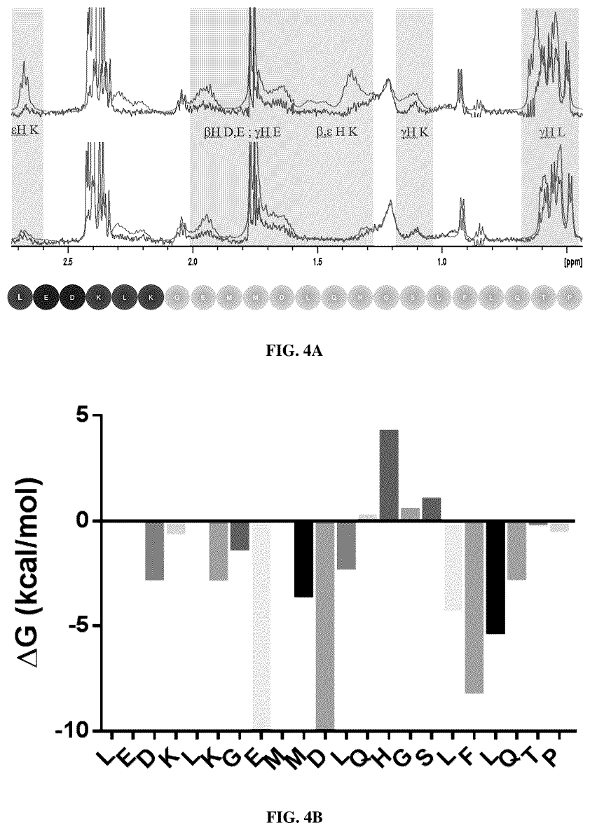

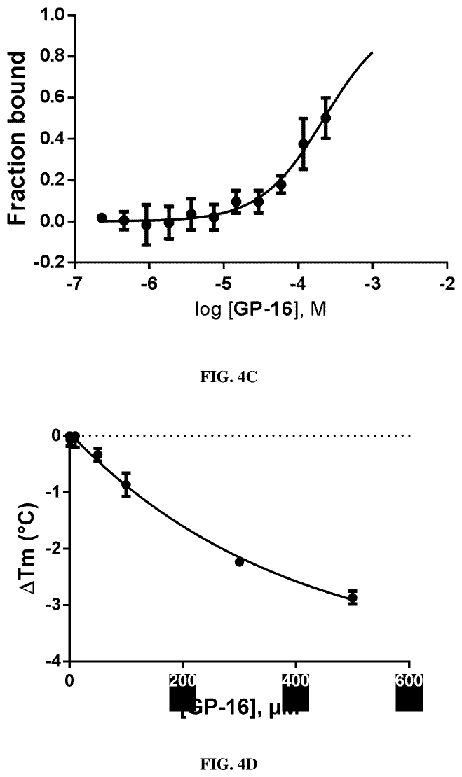

It was next compared polypeptide LP-22 WaterLOGSY and ¹H-NMR spectra. Because WaterLOGSY is a ligand-based NMR spectroscopy that relies on protein-ligand saturation transfer, polypeptide LP-22 residues that do not interact with the protein will be absent of the WaterLOGSY spectrum. A careful comparison between polypeptide LP-22 ¹H and WaterLOGSY spectra highlighted ¹H chemical shifts regions characteristic to lysine, glutamate, aspartate, and leucine aliphatic regions that were not undergoing saturation transfer (FIG. 4A). Calculation of the contribution of each residue to the peptide-binding free energy further suggested that N-terminal residues LEDKLK of polypeptide LP-22, a region rich in those particular amino acids, did not account for much of LP-22 binding energy (FIG. 4B). Removal of these six N-terminal residues led to polypeptide GP-16 (GEMMDLQHGSLFLQTP; SEQ ID NO: 6), a peptide that had a similar WaterLOGSY spectrum (FIG. 4A), thus suggesting that the two polypeptides were interacting very similarly. MST indicated that the interaction with dimeric LDH was slightly weakened, with a Kd=240 μM (FIG. 4C). Consistently, nanoDSF established that polypeptide GP-16 could still destabilize tetrameric LDH in a concentration-dependent manner (EC50=262 μM [142 to 383 μM]) (FIG. 4D).

2.5—Probing Polypeptide GP-16 and LDH-H Tetrameric Interface Hot Spots

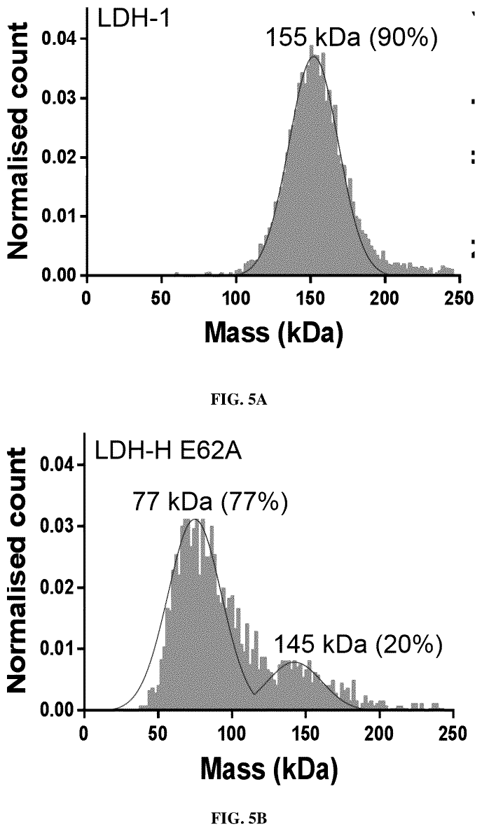

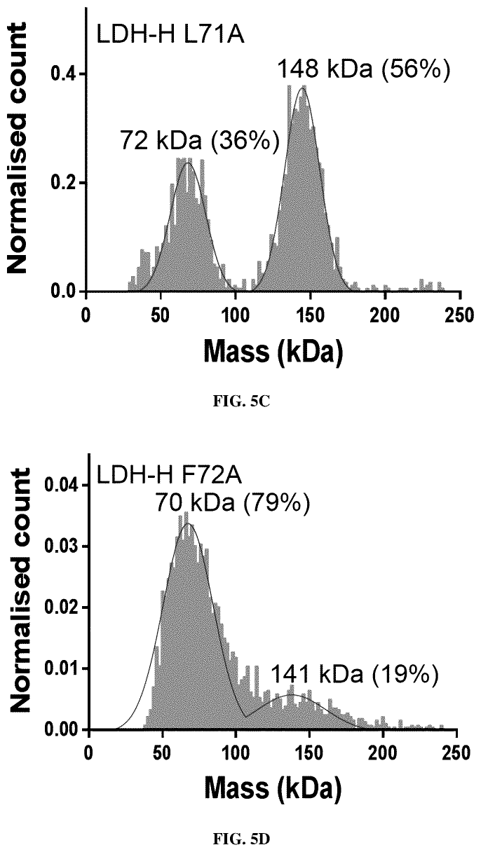

Computational and biophysical data suggested that the polypeptide GP-16 sequence (SEQ ID NO: 6) represents an essential binding region of the LDH tetrameric interface. To verify this hypothesis, the contribution of each residue of cluster B1 was probed to the stability of LDH-H oligomeric state. To that end, an alanine scanning of the LDH-1 sequence (SEQ ID NO: 2) corresponding to polypeptide GP-16 was performed (SEQ ID) NO: 6). The 16 corresponding LDH-H recombinant alanine variants were thus designed, produced, purified and evaluated for their thermal and chemical stability, and by MP (Table 2 and FIG. 5A-D).

TABLE 2
Oligomeric state and stability of LDH-H
variants obtained by Alanine scanning

| | | | | Ratio | |
|---|---|---|---|---|---|
| | Mw | EC₅₀ | Tm | 350/ | SEQ |
| | (kDa) | (M) | (° C.) | 330 nm | ID NO: |
| LDH-1 | 155 ± 17 | 0.953 ± 0.012 | 74.5 ± 0.1 | 1.04 | 2 |
| LDH-Htr | 88 ± 13 | <0.1 | 57.5 ± 0.1 | 0.87 | 4 |
| G61A | 142 ± 10 | 0.735 ± 0.018 | 69.8 ± 0.1 | 1.05 | 52 |
| E62A | 77 ± 18 | <0.1 | 58.9 ± 0.1 | 0.95 | 53 |
| M63A | 141 ± 14 | 0.891 ± 0.012 | 71.6 ± 0.1 | 1.04 | 54 |
| M64A | 149 ± 18 | 0.845 ± 0.010 | 68.7 ± 0.1 | 1.04 | 55 |
| D65A | 143 ± 12 | 0.521 ± 0.012 | 56.4 ± 0.1 | 1.03 | 56 |
| L66A | 137 ± 23 | 0.630 ± 0.011 | 61.1 ± 0.1 | 1.03 | 57 |
| Q67A | 134 ± 16 | 0.893 ± 0.010 | 73.1 ± 0.1 | 1.04 | 58 |
| H68A | 154 ± 15 | 0.619 ± 0.016 | 67.2 ± 0.2 | 1.05 | 59 |
| G69A | 153 ± 18 | 0.722 ± 0.015 | 73.5 ± 0.1 | 1.05 | 60 |
| S70A | 144 ± 13 | 0.580 ± 0.009 | 66.0 ± 0.1 | 1.04 | 61 |
| L71A | 148 ± 12 | <0.1 | 67.1 ± 0.1 | 0.90 | 62 |
| F72A | 70 ± 17 | <0.1 | 53.9 ± 0.1 | 0.94 | 63 |
| L73A | 137 ± 10 | 0.348 ± 0.008 | 62.0 ± 0.1 | 1.03 | 64 |
| Q74A | 143 ± 10 | 0.636 ± 0.020 | 71.3 ± 0.1 | 1.05 | 65 |
| T75A | 145 ± 10 | 0.439 ± 0.012 | 64.9 ± 0.1 | 1.04 | 66 |
| P76A | 142 ± 17 | 0.752 ± 0.011 | 72.2 ± 0.1 | 1.05 | 67 |

Reported values are means ± SEM for melting temperatures and EC50 (n = 6) and means ± SD for the Mw (the values presented here were obtained with MP from one measurement and were repeated at least 3 times with similar results). The reported molecular weights correspond to the main oligomeric state of the protein. Amino acid residue position is calculated with respect to the 1^{st} amino acid residue at the N-terminus of LDH-1 (SEQ ID NO: 2).

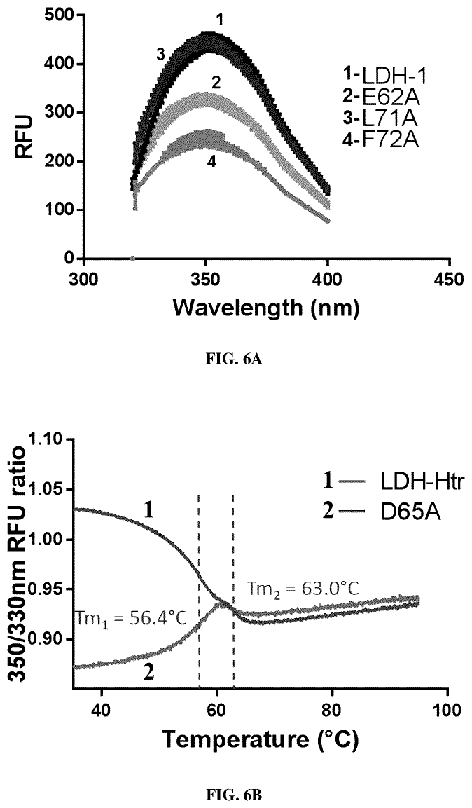

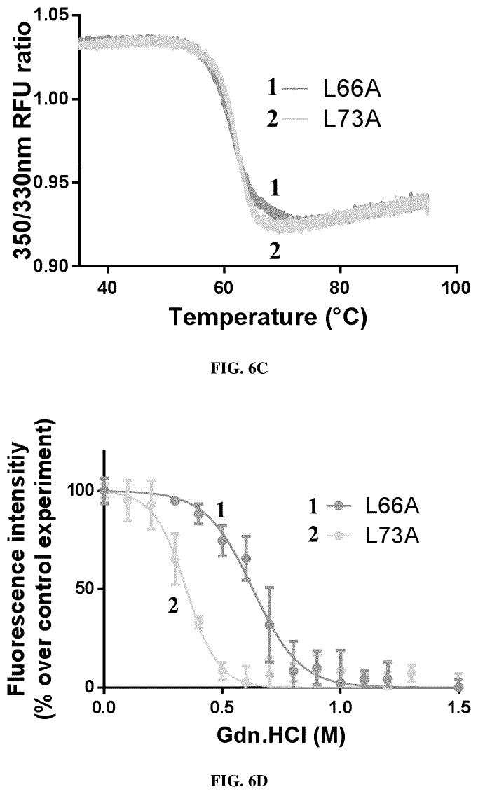

Among the different single-point alanine mutations, three of them significantly impacted the LDH-1 oligomeric state, with variants E62A (SEQ ID NO: 53) and F72A (SEQ ID NO: 63) behaving mainly as dimers in solution, and variant L71A (SEQ ID NO: 62) behaving as a mixture of tetramers and dimers according to MP results (FIG. 5B-D). Consistently, nanoDSF experiments showed that LDH-HF72A (SEQ ID NO: 63) and LDH-HE62A (SEQ ID NO: 53) exhibited denaturation patterns comparable to dimeric LDH-Htr (SEQ ID NO: 4), with a lower initial 350/330 nm ratio and a red-shift instead of the blue-shifts usually observed for tetrameric LDH variants (Table 2). Interestingly, LDH-HL71A (SEQ ID NO: 62) exhibited a lower initial ratio and a red-shift as well, but a Tm 10° C. higher than LDH-Htr. This mixed profile is coherent with the mixture of dimers and tetramers that appear to be present in solution with this variant. Comparison of the tryptophan fluorescence spectra of these variants with LDH-1 showed decays in fluorescence intensity characteristic of the dimeric forms of LDHs for variants E62A and F72A but not L71A (FIG. 6A).

Mutations of L73 (SEQ ID NO: 64) and D65 (SEQ ID NO: 56), two other hot-spots previously suggested by in silico analysis, resulted in tetrameric variants displaying a significant reduction of stability as assessed by both thermal and chemical denaturation (Table 2). In line with the expected reduction of tetrameric stability, dilution experiments of the D65A variant (SEQ ID NO: 56) resulted in concentration-dependent destabilization of the protein and in the apparition of a second unfolding event (FIG. 6B). Interestingly, variants L66A (SEQ ID NO: 57) and L73A (SEQ ID NO: 64) showed similar stabilities by nanoDSF, with Tm of 61.1 and 62.0° C., respectively. However, chemical denaturation experiments demonstrated a striking difference in stability between these two mutants, with EC50s of 0.630 and 0.348 M, respectively (FIG. 6C-D and Table 2). MP experiments validated the presence of an equilibrium between dimers and tetramers for L73A (SEQ ID NO: 64), but not for L66A (SEQ ID NO: 57). In accordance with in silico calculation and available crystallographic data, these results confirm that mutation L73A reduces LDH-1 oligomeric stability. In contrast, mutation L66A appears to impact the stability of the protein differently, for instance, by disturbing its hydrophobic core. These results further highlight the importance of exploiting orthogonal methods when assessing the impact of a mutation on protein stability. Other mutations resulted in tetrameric proteins displaying a medium to low variation of their chemical and thermal stability compared to wild-type LDH-1, highlighting the lesser importance of these residues for the oligomeric state of the protein (Table 2).

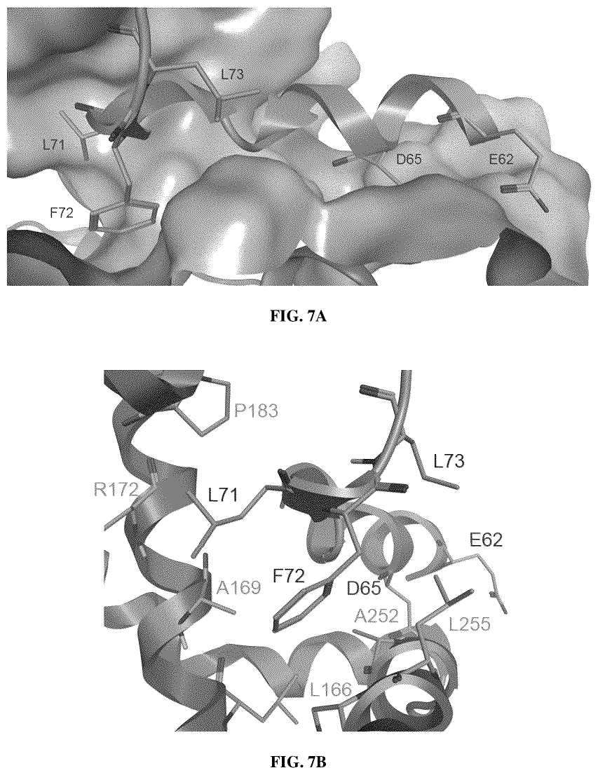

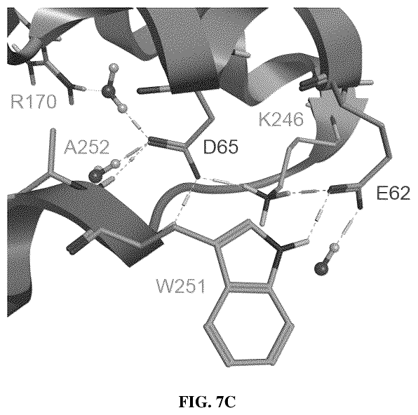

Overall, the different stabilities of the mutants coherently matched with in silico predictions of the AG of interaction, and highlighted new molecular determinants of the LDH tetrameric interface (FIG. 4B). Cluster B1 hot-spots are constituted by the two negatively charged amino-acids, E62 and D65, and by the three consecutive hydrophobic residues: L71, F72 and L73. Based on the crystallographic structure (PDB ID: 1I0Z), E62 and D65 are involved in a hydrogen bond network with water and neighboring residues R170, K246, A252 and W251. L71, F72 and L73 perform hydrophobic interactions between each other and with residues L166, A169, P183, A252 and L255 (FIG. 7A-C). Interestingly, cluster B1 is constituted of both polar and apolar hot spots, which contrasts with the purely lipophilic hot spots that we had previously identified in the LDH tetramerization arm (Thabault et al. (see above)).

3. Conclusion and Discussion

Over the past years, intense efforts were devoted to the development of LDH inhibitors. Unfortunately, the polarity of LDH active site and high intracellular concentrations of the enzyme have challenged the discovery of LDH inhibitors displaying potent and durable in vivo inhibition. Recently, new advances in the development of ligands targeting LDH oligomeric interface have offered new avenues towards LDH inhibition (Thabault et al. (see above); Jafary et al. (Novel Peptide Inhibitors for Lactate Dehydrogenase A (LDHA): A Survey to Inhibit LDHA Activity via Disruption of Protein-Protein Interaction. Sci. Rep. 2019, 9 (4686)); Friberg et al. (Structural Evidence for Isoform-Selective Allosteric Inhibition of Lactate Dehydrogenase A. ACS Omega 2020, 5 (22), 13034-13041)). Targeting protein self-association is an emerging concept in drug design that can bring several advantages over classical orthosteric inhibition. First, targeting the LDH oligomeric interface could unravel new allosteric sites, potentially leading to compounds displaying improved drug-like features compare to LDH active site inhibitors. Secondly, molecules interacting at a protein homomeric interface can lead to its destabilization and degradation, providing compounds with a sub-stoichiometric effect. Here, it was reported the identification and characterization of a new LDH tetrameric interface and its essential residues, using a combination of MP, nanoDSF and chemical stability experiments. Furthermore, it was reported the identification of a family of peptidic ligands that target the tetrameric interface of LDH, destabilize the tetrameric LDH, and stabilize the dimeric LDH-Htr. Altogether, this work provides a structural characterization of the molecular determinant of the LDH tetrameric interface, as well as valuable pharmacological tools for the provision of compounds targeting the LDH oligomeric state.

1-14. (canceled)

15. A polypeptide that inhibits the tetramerization of the LDH subunits, the polypeptide comprising the amino acid sequence of formula (I):
(I)
(SEQ ID NO: 5)
GX₁MMX₂LQHGSX₃X₄X₅QTP
wherein:
X₁ represents amino acid residue E, D, or A;
X₂ represents amino acid residue D, E, or A;
X₃ represents amino acid residue L, A, V, I, F, W, or Y;
X₄ represents amino acid residue F, A, L, V, I, W, or Y;
X₅ represents amino acid residue L, A, V, I, F, W, or Y,
wherein said polypeptide comprises from 16 to 200 amino acid residues, and wherein said amino acid sequence is not SEQ ID NO: 87.

16. The polypeptide according to claim 15, wherein the polypeptide comprises an amino acid sequence selected from the group consisting of SEQ ID NO: 6 to SEQ ID NO: 51.

17. The polypeptide according to claim 15, wherein the polypeptide comprises an amino acid sequence selected from the group consisting of SEQ ID NO: 6 to SEQ ID NO: 28.

18. The polypeptide according to claim 15, wherein the polypeptide comprises an amino acid sequence selected from the group consisting of SEQ ID NO: 6, SEQ ID NO: 8, SEQ ID NO: 10, SEQ ID NO: 11, SEQ ID NO: 17 and SEQ ID NO: 23.

19. The polypeptide according to claim 15, wherein the lactate dehydrogenase subunits are LDH-1 subunits and/or LDH-5 subunits.

20. The polypeptide according to claim 15, wherein the lactate dehydrogenase subunits are LDH-1 subunits.

21. A nucleic acid encoding a polypeptide according to claim 15.

22. A method for preventing and/or treating a disease in an individual in need thereof, comprising at least the step of administering to the individual a therapeutically efficient amount of a polypeptide that inhibits the tetramerization of the LDH subunits, the polypeptide comprising the amino acid sequence of formula (I):
(I)
(SEQ ID NO: 5)
GX₁MMX₂LQHGSX₃X₄X₅QTP
wherein:
X₁ represents amino acid residue E, D, or A;
X₂ represents amino acid residue D, E, or A;
X₃ represents amino acid residue L, A, V, I, F, W, or Y;
X₄ represents amino acid residue F, A, L, V, I, W, or Y;
X₅ represents amino acid residue L, A, V, I, F, W, or Y,
wherein said polypeptide comprises from 16 to 200 amino acid residues, and wherein said amino acid sequence is not SEQ ID NO: 87, or a nucleic acid encoding the same.

23. The method according to claim 22, wherein said disease is a cancer.

24. The method according to claim 23, wherein a further anticancer agent is administered to the individual.

25. The method according to claim 23, wherein the cancer is characterized by metabolic reprogramming.

26. The method according to claim 22, wherein said polypeptide or nucleic acid is to be administered parenterally, subcutaneously, intravenously, or via an implanted reservoir.

27. The method according to claim 22, wherein said polypeptide or nucleic acid is formulated in a sustained release formulation so as to provide sustained release over a prolonged period of time such as over at least 4 weeks.

28. The method according to claim 22, wherein the therapeutically efficient amount of said polypeptide ranges from about 0.001 mg to about 3,000 mg, per dosage unit.
